(19) United States
(12) Patent Application Publication
(10) Pub. No.: US 2024/0148888 A1
(43) Pub. Date: May 9, 2024

(54) INTERCELLULAR ADHESION MOLECULE 1 (ICAM1) ANTIBODY DRUG CONJUGATE AND USES THEREOF

(71) Applicant: Children's Medical Center Corporation, Boston, MA (US)

(72) Inventors: Peng Guo, Natick, MA (US); Jing Huang, Natick, MA (US); Marsha A. Moses, Brookline, MA (US)

(73) Assignee: Children's Medical Center Corporation, Boston, MA (US)

(21) Appl. No.: 18/278,394

(22) PCT Filed: Feb. 23, 2022
(86) PCT No.: PCT/US2022/017499

§ 371 Date: Aug. 22, 2023

Related U.S. Application Data
(60) Provisional application No. 63/152,747, filed on Feb. 23, 2021.

(51) Int. Cl. A61K 47/68; A61K 9/00; A61P 35/00; C07K 16/28

(52) U.S. Cl. CPC A61K 47/68031; A61K 9/0019; A61K 47/6849; A61K 47/6889; A61P 35/00; C07K 16/2821

(57) ABSTRACT

The disclosure provides compositions comprising intercellular adhesion molecule 1 (ICAM1) antibody and methods for using the same for therapeutic applications, for example, treating triple negative breast cancer (TNBC) and predicting drug response.

### RELATED APPLICATIONS

This Application claims the benefit under 35 U.S.C. § 119(e) of U.S. Provisional Application Ser. No. 63/152,747 entitled “INTERCELLULAR ADHESION MOLECULE 1 (ICAM1) ANTIBODY DRUG CONJUGATE AND USES THEREOF,” filed on Feb. 23, 2021, the entire contents of which are incorporated herein by reference.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Feb. 23, 2022, is named C123370193WO00-SEQ-ZJG and is 9,612 bytes in size.

### BACKGROUND

Triple negative breast cancer (TNBC) is a heterogeneous disease, defined by the lack of estrogen receptor (ER), progesterone receptor (PR), and human epidermal growth factor receptor type 2 (HER2). TNBC, which represents 15-20% of all breast cancers, occurs more frequently in women under 50 years of age, in African American women, and in individuals carrying a breast cancer early onset 1 (BRCA1) gene mutation. Due to the lack of therapeutic targets and limited treatment options, the prognosis for TNBC patients remains the poorest among all breast cancer patients.

### SUMMARY

The present disclosure is based, at least in part, on the surprising finding that intercellular adhesion molecule 1 (ICAM1) can be targeted to improve triple negative breast cancer (TNBC) treatment and stratify patient populations for precision medicine. Triple negative breast cancers proliferate independently of signaling mediated by several receptors typically found on the cell surface of other breast cancers, namely estrogen receptor (ER), progesterone receptor (PR), and human epidermal growth factor receptor type 2 (HER2), significantly limiting the range of therapeutic options available for treatment of these types of cancers. Due to these limitations, TNBC are typified by the poorest prognosis among breast cancer types. These limitations are addressed, at least in part, by the present disclosure.

Provided herein, in some aspects, are antibody-drug conjugates (ADCs) that comprise an antibody against intercellular adhesion molecule 1 (ICAM1), which are useful for treatment of TNBC. As described below, use of the ADCs comprising an ICAM1 antibody allowed for preferential targeting of TNBC cells over non-cancerous cells, which can improve the therapeutic window of drugs and limit toxicity. ICAM1 ADCs also display improved specificity for TNBC cells over currently approved ADC therapeutics. Predicting therapeutic sensitivity and responsiveness among patient populations is also challenging given the high genetic heterogeneity of breast cancers. Accordingly, further aspects of the present disclosure provide methods of identifying patient populations for treatment with an ICAM1 antibody or an ADC comprising an ICAM1 antibody in a subject with TNBC.

Aspects of the present disclosure provide methods of treating TNBC comprising administering to a subject in need thereof an effective amount of an antibody drug conjugate (ADC) comprising an intercellular adhesion molecule 1 (ICAM1) antibody conjugated to a drug.

In some embodiments, the drug is selected from the group consisting of: N2′-Deacetyl-N2′-(3-mercapto-1-oxopropyl)mertansine (DM1), N2′-Deacetyl-N2′-(4-mercapto-4-methyl-1-oxopentyl)maytansine (DM4), monomethyl auristatin E (MMAE), monomethyl auristatin F (MMAF). In some embodiments, the drug is MMAE. In some embodiments, the drug is MMAF.

In some embodiments, the ICAM1 antibody and the drug is conjugated via a linker.

In some embodiments, the linker is a cleavable linker. In some embodiments, the cleavable linker is selected from the group consisting of: N-succinimidyl 4-(2-pyridyldithio)pentanoate (SPP), N-succinimidyl 3-(2-pyridyldithio)butanoate (SPDB), Sulfo-SPDB, valine-citrulline (Val-cit), acetyl butyrate, CL2A, and maleimidocaproyl (MC), and Mal-EBE-Mal. In some embodiments, the cleavable linker is MC. In some embodiments, the cleavable linker is Mal-EBE-Mal. In some embodiments, the linker is a non-cleavable linker. In some embodiments, the non-cleavable linker is a selected from the group consisting of N-succinimidyl 4-(N-maleimidomethyl)cyclohexane-1-carboxylate (SMCC) and maleimidomethyl cyclohexane-1-carboxylate (MCC), and MC-VC-PAB. In some embodiments, the non-cleavable linker is MC-VC-PAB.

In some embodiments, the ICAM1 antibody is selected from the group consisting of an IgG, an Ig monomer, a Fab fragment, a F(ab′)2 fragment, a Fd fragment, a scFv, a scAb, a dAb, a Fv, an affibody, a diabody, a single domain heavy chain antibody, and a single domain light chain antibody.

In some embodiments, the ICAM1 antibody is R6.5 or HCD54.

In some embodiments, the ICAM1 antibody is a chimeric antibody. In some embodiments, the ICAM1 antibody is a humanized antibody. In some embodiments, the ICAM1 antibody is a chimerix or humanized version of R6.5 or HCD54.

In some embodiments, the ratio of the ICAM1 antibody and the drug in the ADC is 1:1 to 1:10. In some embodiments, the ratio of the ICAM1 antibody and the drug in the ADC is 1:4.

In some embodiments, the ADC is administered via injection. In some embodiments, the injection is intravenous injection. In some embodiments, the injection is intratumoral injection.

In some embodiments, the ADC is administered at a dosage from 1 mg/kg to 75 mg/kg. In some embodiments, the ADC is administered at a dosage of 5 mg/kg. In some embodiments, the ADC is administered from once every week to once every two months.

In some embodiments, the subject for which an effective amount of ADC is administered is a human subject.

In some embodiments, the type of ICAM1-expressing cancer for which an effective amount of ADC is administered to a subject in need thereof is breast cancer, prostate cancer, ovarian cancer, melanoma, or lung cancer. In some embodiments, the cancer in TNBC.

Further aspects of the present disclosure provide a method of treating TNBC by administering to a subject in need thereof an antibody drug conjugate (ADC) comprising an intercellular adhesion molecule 1 (ICAM1) antibody conjugated to monomethyl auristatin E (MMAE) via a MC-VC-PAB linker.

Further aspects of the present disclosure provide a method of treating TNBC by administering to a subject in need thereof an antibody drug conjugate (ADC) comprising an intercellular adhesion molecule 1 (ICAM1) antibody conjugated to monomethyl auristatin F (MMAF) via a MC linker.

### BRIEF DESCRIPTION OF THE DRAWINGS

The accompanying drawings are not intended to be drawn to scale. In the drawings, each identical or nearly identical component that is illustrated in various FIGs. is represented by a like numeral. For purposes of clarity, not every component may be labeled in every drawing. In the drawings:

FIGS. 1A-1E show differential expression of ICAM1 in human TNBC cells versus normal cells. FIG. 1A: ICAM1 mRNA levels was quantitatively compared in different breast cancer subtypes, molecular subtypes of TNBC, cancer grades of TNBC, and breast tumors with BRCA1/2 or TP53 mutation. *** p<0.001. FIG. 1B: Human TNBC cell surface expression of ICAM1 and TROP2 was compared versus normal MCF10A cells. Non-targeting IgG was used as a control. FIG. 1C: IF staining of ICAM1 and TROP2 on human TNBC cells and normal MCF10A cells. FIG. 1D: Representative imaging flow cytometry images showing cellular internalization of ICAM1 antibodies in human TNBC and normal MCF10A cells. FIG. 1E: Signal intensity analysis for ICAM1 antibody-mediated cell internalization (n=5,000 cells).

FIGS. 2A-2C show selective ablation of human TNBC cells by ICAM1 ADCs. FIG. 2A: Schematic illustration of an ICAM1 ADC. FIG. 2B: DAR characterization of four constructed ICAM1 ADCs including IC1-MMAE, IC1-MMAF, IC1-DM1, and IC1-DM4, by hydrophobic interaction chromatography (HIC). FIG. 2C: In vitro cytotoxicity of four ICAM1 ADCs against a panel of four human TNBC cell lines (MDA-MB-436, MDA-MB-468, MDA-MB-157 and MDA-MB-231) and two non-neoplastic cell lines (MCF10A and HEK293).

FIGS. 3A-3F show tumor-specificity and biodistribution of ICAM1 antibody in nude mice. FIG. 3A: Schematic design of TNBC biodistribution in an immunocompromised nude mouse model. FIG. 3B: In vivo NIR fluorescent images of nude mice at 48 hours after the administration of IgG-Cy5.5, IC1-Cy5.5, or IC1-MMAE-Cy5.5 (n=5 per group). FIG. 3C: Quantified MDA-MB-436 tumor accumulation of IgG-Cy5.5, IC1-Cy5.5, or IC1-MMAE-Cy5.5. * p<0.05, ** p<0.01, NS: not significant. FIG. 3D: Ex vivo NIR fluorescent images of MDA-MB-436 tumors treated by IgG-Cy5.5, IC1-Cy5.5, or IC1-MMAE-Cy5.5. FIG. 3E: Representative ex vivo NIR fluorescent images of six major organs including brain (B), lung (LU), heart (H), liver (L), spleen (S), and kidney (K). FIG. 3F: Quantified normal organ distribution of IgG-Cy5.5, IC1-Cy5.5, or IC1-MMAE-Cy5.5 (n=5).

FIGS. 4A-4D show tumor-specificity and biodistribution of ICAM1 antibody in BALB/c mice. FIG. 4A: Schematic design of tumor biodistribution in an immunocompetent BALB/c mouse model. FIG. 4B: Ex vivo NIR fluorescent images of 4T1 tumors and six normal organs treated by IgG-Cy5.5 and IC1-Cy5.5 (anti-mouse) (n=7 per group). FIG. 4C: Quantified 4T1 tumor and normal organ accumulation of IgG-Cy5.5 and IC1-Cy5.5 (anti-mouse). ** p<0.01, *** p<0.001. NS: not significant. FIG. 4D: Circulating leukocyte uptake of IgG-Cy5.5 and IC1-Cy5.5 (anti-mouse) quantified by flow cytometry. NS: not significant.

FIGS. 5A-5D show ICAM1 ADCs eradicate standard and late-stage TNBC tumors in vivo. FIG. 5A: Schematic design of in vivo efficacy of ICAM1 ADC in standard and late-stage settings of an orthotopic TNBC model. FIG. 5B: Image of excised orthotopic MDA-MB-436 tumors from mice treated with PBS (sham), free Dox, ICAM1 antibody (IC1 Ab), IC1-MMAF or IC1-MMAE in standard setting (n=7-10 per group). FIG. 5C: Tumor progression (left) in standard setting was monitored by tumor volume measurement using a caliper. Tumor mass (center) at end point (day 24) of standard setting was quantified by weight. Mouse body weights (right) in standard setting receiving PBS (sham), free Dox, IC1 Ab, IC1-MMAF or IC1-MMAE. FIG. 5D: Tumor progression (left) receiving PBS (sham), IC1-MMAF or IC1-MMAE in late-stage setting was monitored by tumor volume. Tumor mass (center) at end point (day 34) of late-stage setting was quantified by weight. Mouse body weights (right) in late-stage setting receiving PBS (sham), IC1-MMAF or IC1-MMAE.

FIGS. 6A-6G show ICAM1 ADCs eradicate refractory TNBC tumors in vivo. FIG. 6A: Schematic design of in vivo efficacy of ICAM1 ADCs in a refractory TNBC mouse model. FIG. 6B: Image of excised orthotopic MDA-MB-231 tumors from mice treated with PBS (sham), IC1-MMAF or IC1-MMAE. FIG. 6C: Refractory tumor progression (left) receiving PBS (sham), IC1-MMAF or IC1-MMAE was monitored by tumor volume measurement using a caliper. Tumor mass (center) at end point (day 24) was quantified by weight. Quantified mouse body weights (right) during administration of PBS (sham), free Dox, IC1 Ab, IC1-MMAF, or IC1-MMAE. FIG. 6D: Schematic design of dosage-dependent efficacy of IC1-MMAE in an orthotopic TNBC tumor model. FIG. 6E: Image of excised orthotopic MDA-MB-436 tumors from mice treated with PBS (sham) or IC1-MMAE at three different dosages. FIG. 6F: Tumor progression (left) receiving PBS (sham) or IC1-MMAE at different dosages was monitored by tumor volume. Tumor mass (center) at end point (day 24) was quantified by weight. Quantified mouse body weights (right) during IC1-MMAE administration at different dosages. FIG. 6G: Chronic liver and renal toxicities of IC1-MMAE were analyzed by blood chemistry.

FIGS. 7A and 7B show MTD measurement of IC1-MMAE. FIG. 7A: Schematic design of maximum tolerable dosage of IC1-MMAE in an immunocompetent BALB/c mouse model. FIG. 7B: Quantified mouse bodyweight during MTD test (n=10 per group).

FIG. 8 shows selective ablation of non-TNBC human cancer cells by ICAM1 ADCs. In vitro cytotoxicity of four ICAM1 ADCs (ICAM1-DM4, ICAM1-DM1, ICAM1-MMAE, and ICAM1-MMAF) was tested against a panel of four non-TNBC ICAM1-overexpressing human cancer cell lines: Du145 (prostate cancer), Caov3 (ovarian cancer), A375 (melanoma), and PC9 (lung cancer).

### DETAILED DESCRIPTION OF CERTAIN EMBODIMENTS

### Antibody-Drug Conjugates (ADCs)

### I. ICAM1 Antibodies

Antibody-drug conjugates (ADCs) are a class of immunotherapeutics that comprise an antibody conjugated to a drug. The ADCs of the present disclosure can target cells expressing ICAM1. ICAM1 is a cell surface glycoprotein that has been shown to bind integrins of type CD11a/CD18, or CD11b/CD18 and has been implicated in mediating cell-cell interactions and promoting leukocyte endothelial transmigration. ICAM1 is also referred to as ICAM-1, BB2, Cluster of Differentiation 54 (CD54), and P3.58.

Non-limiting examples of amino acid sequences encoding ICAM1 include UniProtKB Accession Nos. P13597 and P05362.

UniProtKB Accession No. P13597 encodes ICAM1 from Mus musculus has the sequence of:

(SEQ ID NO: 1)
MASTRAKPTLPLLLALVTVVIPGPGDAQVSIHPREAFLPQGGSVQVNCS
SSCKEDLSLGLETQWLKDELESGPNWKLFELSEIGEDSSPLCFENCGTV
QSSASATITVYSFPESVELRPLPAWQQVGKDLTLRCHVDGGAPRTQLSA
VLLRGEEILSRQPVGGHPKDPKEITFTVLASRGDHGANFSCRTELDLRP
QGLALFSNVSEARSLRTFDLPATIPKLDTPDLLEVGTQQKLFCSLEGLF
PASEARIYLELGGQMPTQESTNSSDSVSATALVEVTEEFDRTLPLRCVL
ELADQILETQRTLTVYNFSAPVLTLSQLEVSEGSQVTVKCEAHSGSKVV
LLSGVEPRPPTPQVQFTLNASSEDHKRSFFCSAALEVAGKFLFKNQTLE
LHVLYGPRLDETDCLGNWTWQEGSQQTLKCQAWGNPSPKMTCRRKADGA
LLPIGVVKSVKQEMNGTYVCHAFSSHGNVTRNVYLTVLYHSQNNWTIII
LVPVLLVIVGLVMAASYVYNRQRKIRIYKLQKAQEEAIKLKGQAPPP.

UniProtKB Accession No. P05362 encodes ICAM1 from Homo sapiens and has the sequence:

(SEQ ID NO: 2)
MAPSSPRPALPALLVLLGALFPGPGNAQTSVSPSKVILPRGGSVLVTCS
TSCDQPKLLGIETPLPKKELLLPGNNRKVYELSNVQEDSQPMCYSNCPD
GQSTAKTFLTVYWTPERVELAPLPSWQPVGKNLTLRCQVEGGAPRANLT
VVLLRGEKELKREPAVGEPAEVTTTVLVRRDHHGANFSCRTELDLRPQG
LELFENTSAPYQLQTFVLPATPPQLVSPRVLEVDTQGTVVCSLDGLFPV
SEAQVHLALGDQRLNPTVTYGNDSFSAKASVSVTAEDEGTQRLTCAVIL
GNQSQETLQTVTIYSFPAPNVILTKPEVSEGTEVTVKCEAHPRAKVTLN
GVPAQPLGPRAQLLLKATPEDNGRSFSCSATLEVAGQLIHKNQTRELRV
LYGPRLDERDCPGNWTWPENSQQTPMCQAWGNPLPELKCLKDGTFPLPI
GESVTVTRDLEGTYLCRARSTQGEVTRKVTVNVLSPRYEIVIITVVAAA
VIMGTAGLSTYLYNRQRKIKKYRLQQAQKGTPMKPNTQATPP.

In some embodiments, an ICAM1 protein comprises a sequence that is at least 50%, 55%, 60%, 65%, 70%, 75%, 80%, 85%, 90%, 91%, 92%, 93%, 94%, 95%, 96%, 97%, 98%, 99%, or is 100% identical to SEQ ID NO: 1 or to SEQ ID NO: 2. Additional ICAM1 proteins are well known and may be identified using publically available databases including, e.g., GenBank. An ICAM1 protein may be from any species, including Homo sapiens.

Antibodies of the present disclosure are capable of binding ICAM1. In some embodiments, the ICAM1 antibody is a monoclonal antibody. In some embodiments, the ICAM1 antibody is a polyclonal antibody. In some embodiments, the ICAM1 antibody is a murine antibody. In some embodiments, the ICAM1 antibody is a humanized antibody. In some embodiments, the ICAM1 antibody is a chimeric antibody.

Non-limiting examples of ICAM1 antibodies include clone HCD54 (“HCD54,” commercially available at BioLegend, catalog #322702), UV3, RR1.1, R6.5 (BIRR-1 or Enlimomab, commercially available at Thermo Fisher Scientific, catalog #BMS1011) and BI-505. R6.5 (Enlimomab) is a monoclonal murine antibody produced by ATCC HB-9580 hybridoma cells, e.g., as described in U.S. Pat. No. 5,324,510, which is herein incorporated by reference.

UV3 is a monoclonal antibody and has been shown to bind to ICAM-1 on myeloma cells. In some embodiments, the ICAM1 antibody is a F(ab)′2 fragment of UV3. See, e.g., Huang et al., Hybridoma. 1993 December; 12(6):661-75; and Coleman et al., J Immunother. 2006 September-October; 29(5):489-98, which is each herein incorporated by reference. RR1.1 is a monoclonal ICAM1 antibody. See, e.g., Rothlein and Springer, 1986 J. Exp. Med. 163, 1132-1149, which is herein incorporated by reference. HCD54 is a monoclonal ICAM1 antibody. BI-505 is a fully human ICAM1 monoclonal antibody. See, e.g., Hansson et al., Clin Cancer Res. 2015 Jun. 15; 21(12):2730-6, which is herein incorporated by reference.

The term “bind” refers to the association of two entities (e.g., two proteins). Two entities (e.g., two proteins) are considered to bind to each other when the affinity (K_{D}) between them is <10⁻⁴ M, <10⁻⁵ M, <10⁻⁶ M, <10⁻⁷ M, <10⁻⁸ M, <10⁻⁹ M, <10⁻¹⁰ M, <10⁻¹¹ M, or <10⁻¹² M. One skilled in the art is familiar with how to assess the affinity of two entities (e.g., two proteins).

The term “antibody” encompasses whole antibodies (immunoglobulins having two heavy chains and two light chains), antibody mimetics, and antibody fragments. An “immunoglobulin (Ig)” is a large, Y-shaped protein produced mainly by plasma cells that is used by the immune system to neutralize an exogenous substance (e.g., a pathogens such as bacteria and viruses). Antibodies may be classified as IgA, IgD, IgE, IgG, and IgM. “Antibody fragments” include any antigen binding fragment (i.e., “antigen-binding portion”) or single chain thereof In some embodiments, an “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. In some embodiments, an antibody is an immunoglobulin (Ig) monomer. An antibody may be a polyclonal antibody or a monoclonal antibody.

In some embodiments, an antibody is a heterotetrameric glycoprotein composed of two identical L chains and two H chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of non-limiting examples of different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6, incorporated herein by reference. In some embodiments, an antibody is an IgG.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ and μ, respectively. The γ and α classes are further divided into subclasses on the basis of relatively minor differences in CH sequence and function, e.g., humans express the following subclasses: IgG1, IgG2, IgG3, IgG4, IgA1, and IgA2.

The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, Md. (1991), incorporated herein by reference). The constant domains are not involved directly in binding an antibody to an antigen, but exhibit various effector functions, such as participation of the antibody in antibody dependent cellular cytotoxicity (ADCC).

In some embodiments, the antibody is a monoclonal antibody. A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). Monoclonal antibodies may also be isolated from phage antibody libraries, e.g., using the techniques described in Clackson et al., Nature, 352:624-628 (1991) and Marks et al., J. Mol. Biol., 222:581-597 (1991), incorporated herein by reference.

The monoclonal antibodies described herein encompass “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc.), and human constant region sequences.

In some embodiments, the antibody is a polyclonal antibody. A “polyclonal antibody” is a mixture of different antibody molecules which react with more than one immunogenic determinant of an antigen. Polyclonal antibodies may be isolated or purified from mammalian blood, secretions, or other fluids, or from eggs. Polyclonal antibodies may also be recombinant. A recombinant polyclonal antibody is a polyclonal antibody generated by the use of recombinant technologies. Recombinantly generated polyclonal antibodies usually contain a high concentration of different antibody molecules, all or a majority of (e.g., more than 80%, more than 85%, more than 90%, more than 95%, more than 99%, or more) which are displaying a desired binding activity towards an antigen composed of more than one epitope.

In some embodiments, the antibodies are “humanized” for use in human (e.g., as therapeutics). “Humanized” forms of non-human (e.g., rodent) antibodies are chimeric antibodies that contain minimal sequence derived from the non-human antibody. Humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments, the antibody is an “antibody fragment” containing the antigen-binding portion of a full-length ICAM1 antibody. In some embodiments, an antibody is a single domain heavy chain antibody. In some embodiments, an antibody is a single domain light chain antibody. The antigen-binding portion of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341:544-546, incorporated herein by reference), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. These antibody fragments are obtained using conventional techniques known to those with skill in the art, and the fragments are screened for utility in the same manner as are full-length antibodies.

In some embodiments, an antibody fragment may be a Fc fragment, a Fv fragment, or a single-change Fv fragment. The Fc fragment comprises the carboxy-terminal portions of both H chains held together by disulfides. The effector functions of antibodies are determined by sequences in the Fc region, which region is also the part recognized by Fc receptors (FcR) found on certain types of cells.

The Fv fragment is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

Single-chain Fv also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding (e.g., as described in Pluckthun in The Pharmacology of Monoclonal Antibodies, vol. 113, Rosenburg and Moore eds., Springer-Verlag, New York, pp. 269-315 (1994); Borrebaeck 1995, incorporated herein by reference). In some embodiments, an antibody is a dimerized scFV (a diabody), a scFV timer (a triabody), or a scFV tetrameter (a tetrabody).

Antibodies of the present disclosure include antibody mimetics, including affibody molecules. An affibody is a small protein comprising a three-helix bundle that functions as an antigen binding molecule (e.g., an antibody mimetic). Generally, affibodies are approximately 58 amino acids in length and have a molar mass of approximately 6 kDa. Affibody molecules with unique binding properties are acquired by randomization of 13 amino acids located in two alpha-helices involved in the binding activity of the parent protein domain. Specific affibody molecules binding a desired target protein can be isolated from pools (libraries) containing billions of different variants, using methods such as phage display.

In some embodiments, an ICAM1 antibody binds to an epitope that is present in the extracellular portion of an ICAM1. An “extracellular portion” of an ICAM1 refers to the portion of the ICAM1 that is outside of the cytosol and on the surface of the cell, as opposed to the portion that is inside the cytosol or embedded in the plasma membrane of the cell. The extracellular portion of an ICAM1 typically comprises the glycosylated amino terminal portion of the protein, which mediates cell-cell interactions and promotes leukocyte endothelial transmigration.

Methods of producing antibodies (e.g., monoclonal antibodies or polyclonal antibodies) are known in the art. For example, a polyclonal antibody may be prepared by immunizing an animal, preferably a mammal, with an allergen of choice followed by the isolation of antibody-producing B-lymphocytes from blood, bone marrow, lymph nodes, or spleen. Alternatively, antibody-producing cells may be isolated from an animal and exposed to an allergen in vitro against which antibodies are to be raised. The antibody-producing cells may then be cultured to obtain a population of antibody-producing cells, optionally after fusion to an immortalized cell line such as a myeloma. In some embodiments, as a starting material B-lymphocytes may be isolated from the tissue of an allergic patient, in order to generate fully human polyclonal antibodies. Antibodies may be produced in mice, rats, pigs (swine), sheep, bovine material, or other animals transgenic for the human immunoglobulin genes, as starting material in order to generate fully human polyclonal antibodies. In some embodiments, mice or other animals transgenic for the human immunoglobulin genes (e.g. as disclosed in U.S. Pat. No. 5,939,598), the animals may be immunized to stimulate the in vivo generation of specific antibodies and antibody producing cells before preparation of the polyclonal antibodies from the animal by extraction of B lymphocytes or purification of polyclonal serum.

Monoclonal antibodies are typically made by cell culture that involves fusing myeloma cells with mouse spleen cells immunized with the desired antigen (i.e., hyrbidoma technology). The mixture of cells is diluted and clones are grown from single parent cells on microtitre wells. The antibodies secreted by the different clones are then assayed for their ability to bind to the antigen (with a test such as ELISA or Antigen Microarray Assay) or immuno-dot blot. The most productive and stable clone is then selected for future use.

### II. Drugs

Drugs suitable for use in the ADCs include agents that are therapeutically active against triple negative breast cancer (TNBC). Non-limiting examples of drugs include chemotherapies. In some instances, a drug is a small molecule. In some embodiments, a drug is a cytotoxic small molecule. In some embodiments, a drug is a cytostatic small molecule.

Non-limiting examples of drugs suitable for use in the ADCs include N2′-Deacetyl-N2′-(3-mercapto-1-oxopropyl)mertansine (DM1), monomethyl auristatin E (MMAE), monomethyl auristatin F (MMAF), and duocarmycin, paclitaxel, everolimus, fluorouracil (5-FU), gemcitabine, gemcitabine hydrochloride, mitomycin C, and derivatives thereof. In some embodiments, the drug is maytansine or an analog thereof. In some embodiments, the drug is DM1. DM1 is a cytotoxic maytansine analog that has been shown to inhibit tubulin polymerization. In some embodiments, the maytansine analog is DM4.

The term “small molecule” refers to molecules, whether naturally-occurring or artificially created (e.g., via chemical synthesis) that have a relatively low molecular weight. Typically, a small molecule is an organic compound (e.g., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible.

Any known chemotherapeutic drugs may be used as the drug in the ADC described herein. Non-limiting exemplary chemotherapeutic drugs include: Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, and Vinorelbine.

### III. Linkers

One or more drugs may be conjugated to an ICAM1 antibody using techniques known in the art. In some embodiments, multiple (e.g., 2, 3, 4, 5, 6, 7, 8, 9, 10, or more) drugs are conjugated to an ICAM1 antibody. The ratio of the ICAM1 antibody and the drug in the ADC may be 1:1 to 1:10 (e.g., 1:1, 1:2, 1:3, 1:4, 1:5, 1:6, 1:7, 1:8, 1:9, or 1:10). In some embodiments, the ratio of the ICAM1 antibody and the drug in the ADC is 1:4.

An ICAM1 antibody may be conjugated to a second entity either directly or via a linker. As used herein, “conjugated” or “attached” means two entities are associated, preferably through a covalent bond or with sufficient affinity that the therapeutic or diagnostic benefit of the association between the two entities is realized. In some embodiments, a linker conjugates an ICAM1 antibody to a drug in an ADC. The N-terminus or C-terminus of an ICAM1 antibody may be conjugated to a drug. In some embodiments, a linker can be used to conjugate an ICAM1 antibody to an imaging agent. The N-terminus or C-terminus of an ICAM1 antibody may be conjugated to an imaging agent.

In some embodiments, a linker is a cleavable linker. As used herein, a cleavable linker is capable of releasing a conjugated moiety in response to a stimulus. In some embodiments, the stimulus is a physiological stimulus. Non-limiting examples of stimuli include the presence of an enzyme, acidic conditions, basic conditions, or reducing conditions. For example, cleavable linkers include peptide linkers, β-glucuronide linkers, glutathione-sensitive linkers (or disulfide linkers) and pH-sensitive linkers. In some embodiments, a pH-sensitive linker is cleaved at a pH between 5.0 and 6.5 or between a pH of 4.5 and 5.0. In some embodiments, a pH-sensitive linker is not cleaved when the pH is between 7 and 7.5. In some embodiments, a pH-sensitive linker is not cleaved when the pH is between 7.3 and 7.5. In some embodiments, a cleavable linker is a protease-sensitive linker.

Examples of cleavable linkers include N-succinimidyl 4-(2-pyridyldithio)pentanoate (SPP), N-succinimidyl 3-(2-pyridyldithio)butanoate (SPDB), Sulfo-SPDB, valine-citrulline dipeptide (Val-cit), acetyl butyrate, CL2A, maleimidocaproyl (MC), and Mal-EBE-Mal. In some embodiments, a cleavable linker is a maleimidocaproyl (MC) or Mal-EBE-Mal linker.

See, e.g., Donaghy, MAbs. 2016 May-June; 8(4):659-71, incorporated herein by reference.

In some embodiments, a linker is non-cleavable. In some embodiments, a non-cleavable linker is a linker that is not cleaved within systemic circulation in a subject. In some embodiments, a non-cleavable linker is a linker that is resistant to protease cleavage. Non-cleavable linkers include N-succinimidyl 4-(Nmaleimidomethyl)cyclohexane-1-carboxylate (SMCC), maleimidomethyl cyclohexane-1-carboxylate (MCC), and MC-VC-PAB. In some embodiments, the non-cleavable linker is MC-VC-PAB.

Any of the antibody-drug conjugates may be synthesized using methods known in the art. See, e.g., Yao et al., Int J Mol Sci. 2016 Feb. 2; 17(2). pii: E194.

The ADCs comprising ICAM1 antibody conjugated to a drug are also advantageous to use therapeutically, in part because the drugs (e.g., chemotherapeutic drugs) are toxic and cause severe side effects. By conjugating the drug (e.g., DM1) to the ICAM1 antibody, the toxicity of the ADC may be reduced by at least 20%, at least 30%, at least 40%, at least 50%, at least 60%, at least 70%, at least 80%, at least 90%, at least 99%, compared to the drug in its free from.

### Other ICAM1 Antibody Conjugates

ICAM1 antibodies and/or any of the ADCs of the present disclosure may be conjugated to an imaging agent, which may be useful for predicting the therapeutic sensitivity of a subject with TNBC. For example, imaging agents for computed tomography (CT), positron emission tomography (PET), magnetic resonance imaging (MRI), and endoscopic detection (e.g., endoscopic ultrasound) may be used and can include contrast agents. See, e.g., Bird-Lieberman et al., Nat Med. 2012; 18(2):315-21; Van den Brande et al., Gut. 2007; 56(4):509-17, which is each herein incorporated by reference. In some embodiments, the contrast agent is administered as a salt. In some embodiments, the imaging agent is a gadolinium-based MRI contrast agent. For example, an imaging agent may be a gadolinium-diethylenetriamine pentaacetic acid (Gd-DTPA or DTPA-Gd). See, e.g., Carr et al., AJR Am J Roentgenol. 1984 August; 143(2):215-24.

One or more imaging agents may be conjugated to an ICAM1 antibody or an ADC described herein using techniques known in the art. In some embodiments, multiple (e.g., e.g., 2, 3, 4, 5, 6, 7, 8, 9, 10, or more) imaging agents are conjugated to an ICAM1 antibody. The ratio of the ICAM1 antibody or ADC and the imaging agent may be 1:1 to 1:10 (e.g., 1:1, 1:2, 1:3, 1:4, 1:5, 1:6, 1:7, 1:8, 1:9, or 1:10). In some embodiments, the ratio of the ICAM1 antibody or ADC and the imaging agent is 1:4. Any of the linkers disclosed herein may be used to conjugate an imaging agent to an ICAM1 antibody or to an ADC described herein.

An imaging agent may be visualized with a suitable detection method (e.g., by CT, PET, MM, ultrasound, and/or endoscopic detection).

### Pharmaceutical Compositions and Uses Thereof

Compositions comprising any of the ADCs or other ICAM1 antibody conjugates disclosed herein are encompassed by the present disclosure. In some embodiments, the composition is formulated as a pharmaceutical composition for administration to a subject.

A subject may have, be suspected of having, or be at risk for triple negative breast cancer (TNBC). Breast cancers are classified based on the receptor proteins expressed or not expressed on the cell surface of breast cancer cells. TNBC cells are characterized as breast cancer cells without cell surface expression of estrogen receptor (ER), progesterone receptor (PR), and human epidermal growth factor receptor 2 (HER2). In contrast, ER+ breast cancer cells express estrogen receptors at their cell surface, while HER2+ breast cancer cells express HER2 at their cell surface.

TNBC may also be stratified based on whether or not the cancer has metastasized. TNBC may be classified as stage 0 (carcinoma in situ), stage I, stage II, stage III, stage IV, or stages therewithin (e.g., stage IIA, stage IIB, etc.). A non-limiting staging method is the TNM system, which evaluates the extent of the tumor (T), the spread of the cancer to nearby lymph nodes (N), and whether the cancer has spread to distant sites (M). The various T, N, and M levels (e.g., Table 1) may then be used to determine the stage of TNBC cancer (e.g., Table 2). Tables 1-2 show TNBC classification based on the Eighth Edition of the AJCC/UICC TNM staging system and as described by Cong et al. Sci Rep. 2018 Jul. 10; 8(1):10383.

TABLE 1
Non-limiting examples of TNM staging definitions

| | |
|---|---|
| T1 | Maximum tumor diameter ≤2 cm |
| T2 | Maximum tumor diameter >2, ≤4 cm |
| T3 | Maximum tumor diameter >4 cm |
| T4 | Tumor involves the celiac axis, common |
| N0 | No regional lymph node metastasis |
| N1 | Metastasis in 1-3 regional lymph nodes |
| N2 | Metastasis in ≥4 regional lymph nodes |
| M0 | No distant metastasis |
| M1 | Distant metastasis |

TABLE 2
TNBC Staging Levels

| | T | N | M |
|---|---|---|---|
| IA | T1 | N0 | M0 |
| IB | T2 | N0 | M0 |
| IIA | T3 | N0 | M0 |
| IIB | T1-T3 | N1 | M0 |
| III | T4 | any N | M0 |
| IV | any T | any N | M1 |

In some embodiments, a subject may have, be suspected of having, or be at risk for a cancer other than TNBC. Non-limiting examples of other cancers include breast cancer, prostate cancer, ovarian cancer, melanoma, lung cancer, and pancreatic cancer. In some embodiments, the breast cancer is not TNBC. Other cancers may also be stratified based on whether or not the cancer has metastasized and may be classified as stage 0 (carcinoma in situ), stage I, stage II, stage III, stage IV, or stages therewithin (e.g., stage IIA, stage IIB, etc.). Other cancers may also be classified using the TNM staging method, in which the various T, N, and M levels are used to determine the cancer stage (e.g., Tables 1 and 2).

In some embodiments, any of the pharmaceutical compositions disclosed herein comprising an imaging agent is administered in an effective amount to a subject to determine the level of ICAM1 in a tumor of a subject with TNBC or another cancer (e.g., CT, PET, MRI, and endoscopic detection (e.g., endoscopic ultrasound)). The imaging methods for determining the level of ICAM1 described herein are advantageous compare to conventional methods (e.g., biopsy and analyzing the tissue obtained from the biopsy). The imaging methods (e.g., MRI) is non-invasive, and provides a comprehensive view of the tumor for ICAM1 level, providing more accurate assessment of the tumor for prediction of outcome and/or responsiveness to treatment (e.g., treatment with ICAM1 antibody or ADC comprising ICAM1 antibody).

In some embodiments, the level of ICAM1 is detected in a subject with TNBC or another cancer who has been administered a pharmaceutical composition of the present disclosure comprising an ICAM1 antibody and an imaging agent. In some embodiments, the ICAM1 level detected in the tumor of the subject is at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% higher than a control. In some embodiments, the ICAM1 level detected in the tumor of the subject is substantially similar to the control.

In some embodiments, a control is a subject with a tumor having a known level of ICAM1. In some embodiments, a control is the level of ICAM1 in the breast tissue of a subject who does not have a tumor. In some embodiments, a control is a subject with a tumor having a low level of ICAM1. In some embodiments, a low level of ICAM1 is not detectable. In some embodiments, a control is a subject with a tumor having a high level of ICAM1. In some embodiments, a high level of ICAM1 is at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% higher than the level of ICAM1 detected in breast tissue of a healthy subject.

In some embodiments, the level of ICAM1 detected in a tumor using a method disclosed herein is predictive of a subject with TNBC or another cancer responding to treatment with an ICAM1 antibody or an antibody drug conjugate (ADC) comprising an intercellular adhesion molecule 1 (ICAM1) antibody conjugated to a drug. In some embodiments, a higher level of ICAM1 detected in a tumor as compared to the tumor of a subject with a lower level of ICAM1 is predicted to be more responsive to treatment with an ICAM1 antibody or an ADC disclosed herein. In some embodiments, a subject with a higher level of ICAM1 in a tumor as compared to a subject with a lower level of ICAM1 in a tumor is at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% more responsive to treatment with a composition comprising an ICAM1 antibody (e.g., an ICAM1 ADC and/or an ICAM1 antibody not conjugated to a drug) In some embodiments, a method disclosed herein comprises administering an ICAM1 antibody or an ADC antibody disclosed herein after identifying the subject as being responsive.

In some embodiments, the level of ICAM1 detected in a tumor using a method disclosed herein is indicative of the stage of cancer. In some embodiments, the level of ICAM1 detected in a tumor is indicative of stage 0, stage I, stage II, stage III, or stage IV.

Without being bound by a particular theory, in some embodiments, administration of an ICAM1 antibody conjugated to an imaging agent or an ICAM1 ADC conjugated to an imaging agent may serve a dual purpose of visualizing a tumor and treating the tumor.

In some embodiments, administration of an ICAM1 antibody and/or an ADC comprising an ICAM1 antibody or a pharmaceutical composition thereof inhibits the growth of a tumor. In some embodiments, administration of an ICAM1 antibody and/or an ADC comprising an ICAM1 antibody or a pharmaceutical composition thereof results in regression of a tumor. In some embodiments, administration of an ICAM1 antibody and/or an ADC comprising an ICAM1 antibody or a pharmaceutical composition thereof decreases the size of a tumor by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% as compared to a control. In some embodiments, the control is a subject who has not been treated with a composition that comprises an ICAM1 antibody.

In some embodiments, administration of an ICAM1 antibody and/or an ADC comprising an ICAM1 antibody or a pharmaceutical composition thereof disclosed herein decreases proliferation by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% higher than a control. In some embodiments, proliferation is measured using Ki67 staining. In some embodiments, the control is a subject who has not been treated with a composition that comprises an ICAM1 antibody.

In some embodiments, administration of an ICAM1 antibody and/or an ADC comprising an ICAM1 antibody or a pharmaceutical composition thereof disclosed herein decreases metastasis of a tumor by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% as compared to a control. In some embodiments, the control is a subject who has not been treated with a composition that comprises an ICAM1 antibody.

In some embodiments, administration of an ICAM1 antibody and/or an ADC comprising an ICAM1 antibody or a pharmaceutical composition thereof disclosed herein does not decrease the viability of healthy cells. In some embodiments, administration of an ADC or a pharmaceutical composition comprising an ADC disclosed herein allows for the effective amount (e.g., concentration) of a drug to be lower than if the drug was not conjugated to an ICAM1 antibody. In some embodiments, the effective amount of a drug is lowered by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, or at least 1,000% as compared to administration of the drug alone.

In some embodiments, the pharmaceutical composition further comprises a pharmaceutically acceptable carrier. “Pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” may be a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the pharmaceutical composition may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

In some embodiments, the pharmaceutical compositions used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

“A therapeutically effective amount” or “effective amount” as used herein refers to the amount of each therapeutic agent (e.g., therapeutic agents for treating any of cancers described herein) of the present disclosure required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons.

Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system. Frequency of administration may be determined and adjusted over the course of therapy, and is generally, but not necessarily, based on treatment and/or suppression and/or amelioration and/or delay of a disease. Alternatively, sustained continuous release formulations of a polypeptide may be appropriate. Various formulations and devices for achieving sustained release are known in the art.

In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the anti-cancer agent used) can vary over time.

In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the anti-cancer agent (such as the half-life of the anti-cancer agent, and other considerations well known in the art).

For the purpose of the present disclosure, the appropriate dosage of a therapeutic agent as described herein will depend on the specific agent (or compositions thereof) employed, the formulation and route of administration, the type and severity of the disease, whether the anti-cancer agent is administered for preventive or therapeutic purposes, previous therapy, the subject's clinical history and response to the antagonist, and the discretion of the attending physician. Typically, the clinician will administer an anti-cancer agent until a dosage is reached that achieves the desired result. Administration of one or more anti-cancer agents can be continuous or intermittent, depending, for example, upon the recipient's physiological condition, whether the purpose of the administration is therapeutic or prophylactic, and other factors known to skilled practitioners. The administration of an anti-cancer agent may be essentially continuous over a preselected period of time or may be in a series of spaced dose, e.g., either before, during, or after developing a disease.

As used herein, the term “treating” refers to the application or administration of an anti-cancer agent to a subject in need thereof. “A subject in need thereof”, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In some embodiments, the non-human animal is a mammal (e.g., rodent, e.g., mouse or rat), primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey). The non-human animal may be a male or female at any stage of development. The non-human animal may be a transgenic animal or genetically engineered animal.

The methods described herein may be used to treat ICAM1-expressing cancer. In some embodiments, the ICAM-1 expressing cancer is breast cancer, prostate cancer, ovarian cancer, melanoma, or lung cancer. In some embodiments, the ICAM-1 expressing cancer is not TNBC.

In some embodiments, the subject is a companion animal (e.g. a pet or service animal). “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include rodents (e.g., rats, mice, guinea pigs, and hamsters), rabbits, or non-human primates.

Alleviating a disease (e.g., cancer) includes delaying the development or progression of the disease or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease includes initial onset and/or recurrence.

Conventional methods, known to those of ordinary skill in the art of medicine, can be used to administer the pharmaceutical composition the subject, depending upon the type of disease to be treated or the site of the disease. The pharmaceutical composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In some embodiments, the pharmaceutical composition is administered via intravenous injection or infusion. In addition, it can be administered to the subject via injectable depot routes of administration such as using 1-, 3-, or 6-month depot injectable or biodegradable materials and methods. In some embodiments, the pharmaceutical composition is administered via injection. In some embodiments, injection is intravenous injection or intratumoral injection.

### EXAMPLES

### Example 1: Development of Antibody Drug Conjugates (ADCs) Targeting ICAM1

### Introduction

Triple negative breast cancer (TNBC) is a heterogeneous disease, defined by the lack of estrogen receptor (ER), progesterone receptor (PR), and human epidermal growth factor receptor type 2 (HER2). Late-stage and refractory TNBC represents a major challenge to the improvement of clinical outcomes for breast cancer patients. In 2021, over 28,000 patients were estimated to be diagnosed with TNBC in the United States, representing 10-15% of breast cancer incidence¹⁻³. TNBC is more prevalent in non-Hispanic black women, young women under the age of 40 and women carrying breast cancer early onset 1 or 2 (BRCA1/2) gene mutations^{4, 5}. Moreover, patients with late-stage or refractory TNBC tumors respond poorly to first line chemotherapy and do not respond to established hormone and HER2-targeted therapeutics due to the lack of expression of these molecular targets thereby dramatically exacerbating their poor clinical outcomes^{2, 3, 6}. The prognosis for TNBC patients remains the poorest among all breast cancer patients, as the current 5 year survival rate of TNBC patients is less than 77%, significantly lower than the more than 90% survival rate for patients with other types of breast cancer⁷⁻⁹.

Although several second-line therapeutics including poly(ADP)-ribose polymerase (PARP) inhibitors (e.g., Olaparib and Talazoparib) and immune checkpoint inhibitors (e.g., Atezolizumab and Pembrolizumab) have been approved for the treatment of TNBC, the clinical benefits of these therapeutics are limited to small subsets (˜20-30%) of TNBC patients, due to limited BRCA1/2 mutation rates or PD-L1 expression¹⁰⁻¹². Novel TNBC-targeted therapeutics with different mechanisms of action that can work in synergy with these approved modalities are likely to further increase clinical efficacy¹³⁻¹⁵. Antibody drug conjugates (ADCs) are an emerging class of immuno-chemotherapeutics featuring the structure of a monoclonal antibody conjugated to cytotoxic agents via chemical linkers. The monoclonal antibody of an ADC functions as a tumor-homing ligand that guides its conjugated agent to selectively ablate antigen-overexpressing tumors while sparing normal tissues. Compared to T-cell immunotherapy (e.g., chimeric antigen receptor-T cell (CAR-T) or immune checkpoint blockade) or nanomedicines (e.g., liposomes), ADCs feature superior tumor tissue penetration due to their ultrasmall size (<10 nm), which is ˜1,000 fold smaller than the size of a T-cell, creating an attractive opportunity to increase drug delivery into tumors. ADCs have demonstrated promising clinical efficacy in various cancers¹³, including aggressive solid tumors that respond poorly to T-cell immunotherapy. For instance, sacituzumab govitecan, a trophoblast antigen 2 (TROP2)-targeted ADC, was approved for treating refractory TNBC patients with a ˜33% response rate and a median progression-free survival of 5.5 months¹⁶. Given the poor prognosis and unimpressive efficacy of current TNBC treatment modalities, there remains an urgent and unmet need to identify new druggable targets and to develop more effective ADCs for TNBC therapy.

ICAM1, also called CD54, is a transmembrane cell surface glycoprotein that regulates intercellular adhesion during inflammatory injury, viral infection and tumorigenesis¹⁷. ICAM1 is aberrantly overexpressed in multiple types of cancers and is frequently associated with an aggressive phenotype and worse prognosis. ICAM1 has previously been identified as a TNBC cell surface marker through unbiased quantitative screening of G protein-coupled receptor (GPCR) signaling proteins^{18, 19}. ICAM1 was found to be highly enriched on the cancer cell surface of human TNBC tumors relative to normal mammary tissues¹⁸⁻²². The NFkB pathway has likewise been implicated in aberrantly upregulating ICAM1 expression in cancer cells by binding to the ICAM1 promoter and causing its hyperactive transcription^{23, 24}. An ICAM1 neutralizing antibody has been investigated as a potential cancer therapeutic, however blocking ICAM1 signaling cascades alone has not yielded satisfying clinical efficacy²⁵⁻²⁷. Thus, as described herein, ICAM1 ADCs that induces potent and durable tumor regression in vivo have been developed. It was hypothesized that a rationally designed ICAM1 ADC would precisely target and eradicate TNBC tumors while sparing healthy organs and tissues. To test this hypothesis, a panel of four ICAM1 ADCs with different chemical linkers and cytotoxic agents were constructed and evaluated in vitro against multiple TNBC cell types. The in vivo efficacy of these ADCs was also tested in a series of standard, late-stage and refractory TNBC models.

### Results and Discussion

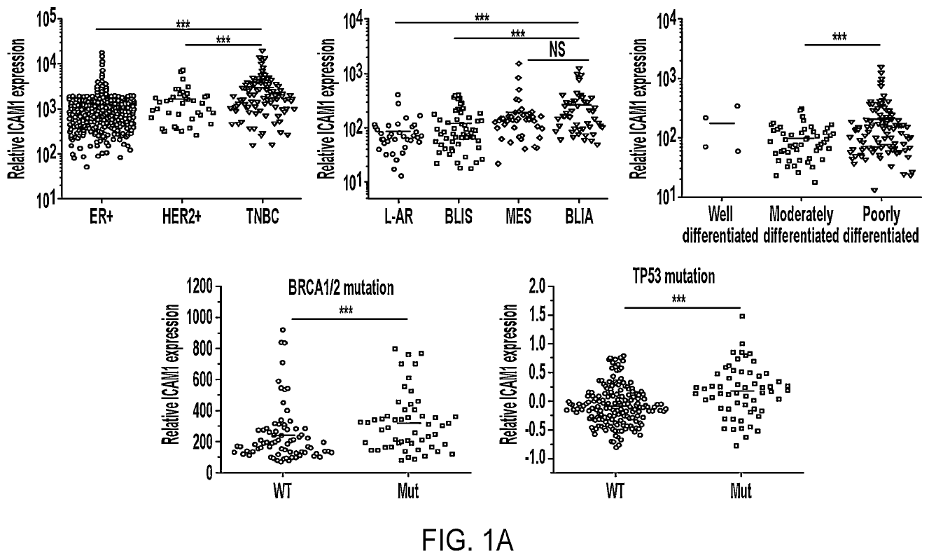

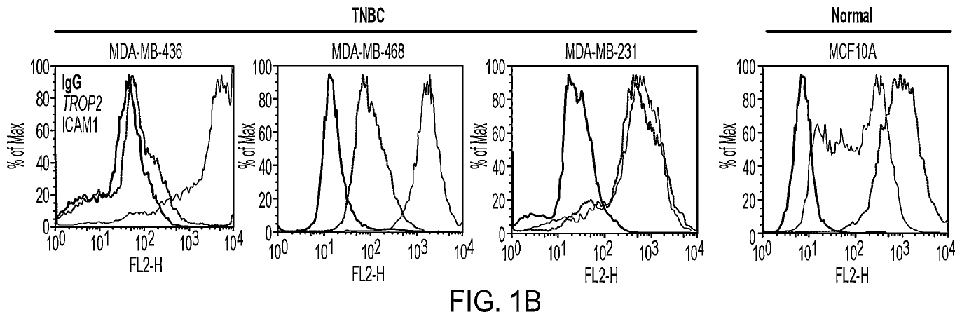

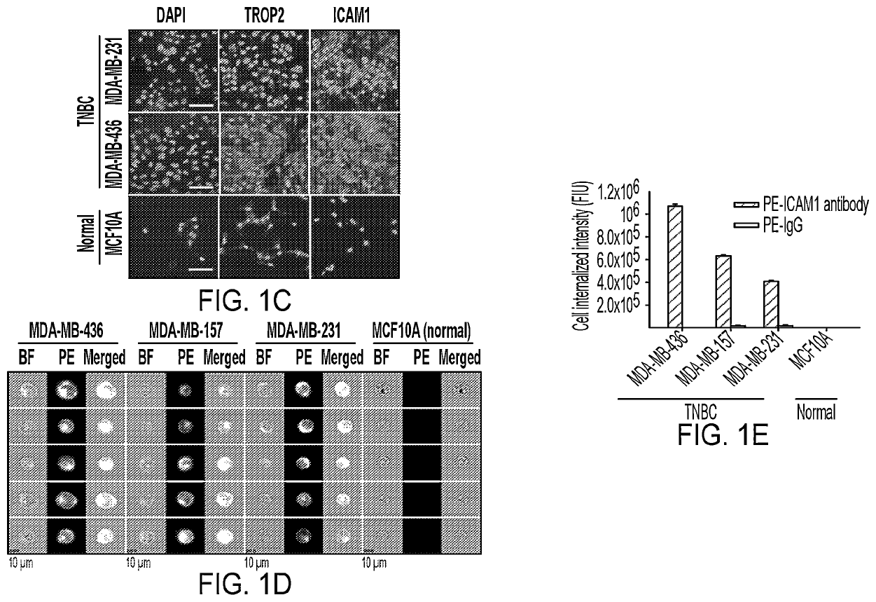

ICAM1 was previously identified as a novel molecular target for TNBC by measuring protein expression in a cohort of 149 cases of human breast tumor tissues along with 144 human normal tissues including breast and 19 other organs using immunohistochemistry (IHC)¹⁸. ICAM1 staining was found to be present at a significantly greater level in cancer cells of TNBC tumors, as compared to other breast cancer subtypes, and is completely absent in normal mammary tissues. Genomic analyses were used to further to interrogate the relationship of ICAM1 expression with more detailed TNBC characteristics, including its molecular subtype, tumor differentiation, and oncogene mutation status, by querying the R2: Genomics Analysis and Visualization Platform (hgserver1.amc/nl). ICAM1 mRNA expression was quantitatively compared in three breast cancer subtypes: ER-positive, HER2-positive, and TNBC (FIG. 1A). Consistent with pathological staining results¹⁸, ICAM1 mRNA levels in TNBC tumor tissues (n=116) are significantly higher than those of HER2-positive (n=40) and ER-positive (n=808) breast tumors. Given that TNBC has recently been identified as a heterogeneous disease classified into four distinct molecular subtypes (basal-like immunosuppressed (BLIS), basal-like immunoactivated (BLIA), luminal androgen receptor (L-AR) and mesenchymal (MES)²⁸), ICAM1 mRNA levels were also assessed in these molecular subtypes. BLIA (n=54) showed the highest level of ICAM1 expression, followed by MES (n=47), L-AR (n=60), and BLIS (n=37). The correlation between ICAM1 expression and TNBC tumor differentiation was also analyzed, and it was found that ICAM1 expression is significantly higher within poorly differentiated TNBC tumors (grade 3, n=108), as compared to moderately differentiated tumors (grade 2, n=53). It is generally known that poorly differentiated TNBC tumors are strongly associated with poorer prognoses, thus ICAM1 may serve as a potential biomarker for predicting clinical outcomes of TNBC patients. The relationship between ICAM1 expression and cancer mutation status was also examined, and it was found that ICAM1 levels positively correlate with BRCA1/2 or TP53 mutations, which occur in 20% and 80% of TNBC tumors, respectively²⁹⁻³¹.

Given that TROP2 is a new clinically approved ADC target for TNBC therapy¹⁶, it was selected as a positive control for the evaluation of potential ICAM1 ADCs. The cell surface expression of ICAM1 and TROP2 in three established human TNBC cell lines (MDA-MB-436, MDA-MB-157 and MDA-MB-231) was examined, along with normal human mammary epithelial MCF10A cells (FIG. 1B). The cell surface expression of ICAM1 is 11- to 35-fold higher than TROP2 on two TNBC cell lines (MDA-MB-436 and MDA-MB-468) and is equivalent in the third cell line (MDA-MB-231). More importantly, ICAM1 surface expression on normal MCF10A cells is 7-fold lower than that of TROP2. These findings were confirmed by immunofluorescent (IF) staining of ICAM1 and TROP2 in the same cells (FIG. 1C). The subcellular localization of ICAM1 is predominantly on the plasma membranes of TNBC cells and is undetectable on plasma membranes of normal MCF10A cells. These results provide evidence that ICAM1 is more highly overexpressed and is more tumor-specific than TROP2 in human TNBC cells, which could potentially contribute to a higher tumor-specificity and fewer on-target/off-tumor adverse effects during in vivo delivery of ADCs targeting ICAM1.

Cellular internalization is another important factor affecting ADC efficacy. In order to determine whether ICAM1 ADCs can selectively enter TNBC cells and rapidly traffic into endosomes for release of cytotoxic agents, the cellular internalization rates of ICAM1 antibody were examined in multiple TNBC cell lines, as well as normal MCF10A cells. Confocal fluorescent images revealed that ICAM1 antibodies were rapidly internalized by all three TNBC cell lines examined (MDA-MB-436, MDA-MB-157 and MDA-MB-231) after a 1-hour incubation (FIG. 1D). The internalized ICAM1 antibodies are aggregated, rather than dispersed, in the cytoplasm of TNBC cells, indicating that they are trafficked into endosomes/lysosomes by receptor-mediated endocytosis^{32, 33}. In contrast, no internalization was observed in normal MCF10A cells due to their lack of ICAM1 antigen expression. Cellular internalization rates of ICAM1 antibody in three TNBC cell lines were approximately 42 to 109-fold higher than those in MCF10A cells, and approximately 21 to 129-fold higher than those of observed with a non-targeting IgG (FIG. 1E). These results confirm that ICAM1 is highly overexpressed in TNBC cells and that it facilitates efficient cellular internalization of bound antibodies, suggesting that it has outstanding potential utility as an ADC target for treating TNBC.

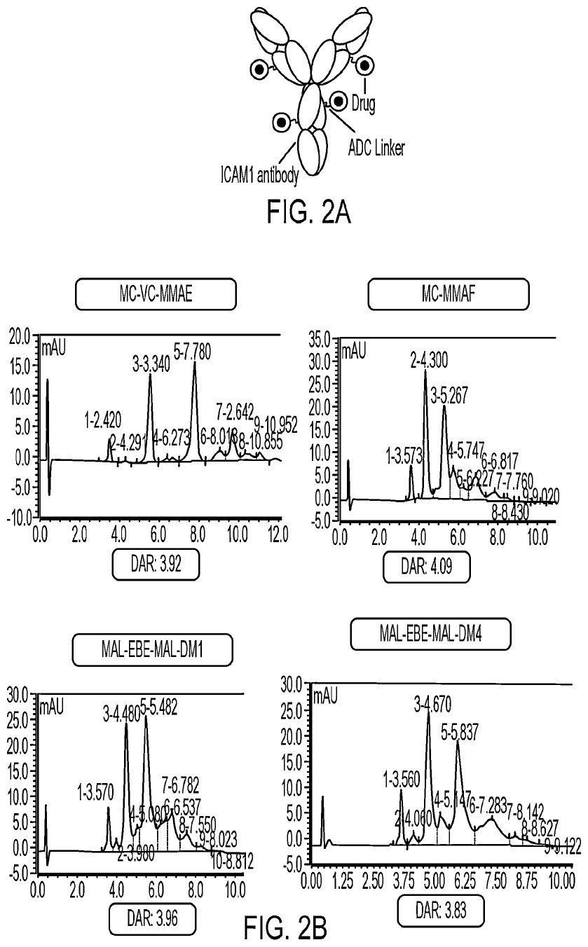

To identify the optimal ADC formulation for TNBC, a panel of ICAM1 ADCs was designed and constructed by conjugating a monoclonal human/mouse chimeric ICAM1 antibody (clone #R6.5)³⁴ with cytotoxic drugs (FIG. 2A) to produce four different ADC combinations, namely MC-Vc-Pab-MMAE, MC-MMAF, Mal-EBE-Mal-DM1, and Mal-EBE-Mal-DM4. The ICAM1 ADCs were named according to their antibodies and conjugated drugs, as follows: ICAM1 antibody conjugated to MC-Vc-Pab-MMAE (IC1-MMAE); ICAM1 antibody conjugated to MC-MMAF (IC1-MMAF); ICAM1 antibody conjugated to Mal-EBE-Mal-DM1 (IC1-DM1); and ICAM1 antibody conjugated to Mal-EBE-Mal-DM4 (IC1-DM4). This ICAM1 antibody selection is based on the excellent human safety profile of human/mouse chimeric ICAM1 antibody (clone #R6.5), which has been previously determined in Phase I/II clinical trials^{27, 34}. The choice of ADC linker-drug was then examined. MC-VC-Pab-MMAE features an enzyme-cleavable linker that can be selectively cleaved by cathepsin B proteases in cell endosomes/lysosomes, resulting in a rapid drug release from ADC antibodies upon cellular internalization while maintaining ADC integrity during blood circulation¹³⁻¹⁵. The other MC and Mal-EBE-Mal linkers are non-cleavable thus the drug release depends on lysosomal degradation of the conjugated antibody. Both enzyme-cleavable and non-cleavable linkers are currently used in clinically-approved ADCs for treating different cancers¹³⁻¹⁵. All four drugs utilized in the examined ICAM1 ADCs are potent microtubule inhibitors with cytotoxicities that are approximately 100 to 1,000-fold higher than standard-of-care chemodrugs (e.g., paclitaxel or gemcitabine)¹³⁻¹⁵. MMAE, MMAF and DM1 are clinically utilized ADC drugs and DM4 has also been clinically investigated as an ADC drug for treating TNBC. However, it remains unclear which ADC linker-drug combination is most effective for TNBC treatment in the absence of an unbiased quantitative screen. Moreover, the drug to antibody ratio (DAR), defined as the number of drug molecules conjugated per antibody, is another important factor affecting ADC efficacy and human safety. A DAR value of 4.0 was well-established for microtubule inhibitor ADC drugs in multiple clinically-approved ADCs (e.g., trastuzumab emtansine and brentuximab vedotin) demonstrating excellent clinical efficacy and human safety profiles¹³⁻¹⁵. Thus, the DAR of the four constructed ICAM1 ADCs was adjusted to an equivalent value of 4 by controlling the antibody/drug input ratios during the ADC conjugation. The resulting DAR values of four ICAM1 ADCs were determined by hydrophobic interaction chromatography (HIC) as being 3.96 for IC1-DM1, 3.83 for IC1-DM4, 3.92 for IC1-MMAE, and 4.09 for IC1-MMAF, respectively (FIG. 2B).

The in vitro cytotoxicity of the four ICAM1 ADCs was then assessed against a panel of TNBC cell lines, and half maximum inhibitory concentrations (IC50s) were quantified using a Dojindo assay. Among the four ICAM1 ADCs, exposure to IC1-MMAE and IC1-MMAF elicited a significantly higher in vitro efficacy as compared to IC1-DM1 and IC-DM4 in three of the four tested TNBC cell lines (FIG. 2C). The IC50s of IC1-MMAE and IC1-MMAF were determined to be 13.1 and 7.3 pM for MDA-MB-436, 250.0 and 68.7 pM for MDA-MB-468, 221.7 and 116.0 pM for MDA-MB-157, respectively. It is noteworthy that MDA-MB-436 and MDA-MB-157 cells represent both Caucasian and African American TNBC cell lines, respectively. In contrast, the IC50s in IC1-DM1 and IC1-DM4 are at least 5-fold higher. Notably, the IC50s of IC-MMAE and IC-MMAF are approximately 1,000-fold lower than Doxorubicin, a standard-of-care chemodrug used in TNBC treatment. It was observed that MDA-MB-231 cells exhibited strong drug resistance to all four tested ICAM1 ADCs. Due to the fact that MDA-MB-231 cells were also reported to be resistant to sacituzumab govitecan, the clinically used TROP2 ADC for TNBC³⁵, this TNBC cell line was selected for the generation of an ADC-resistant TNBC model in the following animal study. The cytotoxicity of ICAM1 ADCs was also evaluated in non-neoplastic MCF10A and HEK293 cells, both of which lack ICAM1 antigen expression. As expected, no cytotoxicity was observed in ICAM1-negative MCF10A and HEK293 cells until a very high ADC concentration of over 6.7 nM was reached thereby, indicating that the potency of the ADCs in ablating TNBC cells is dependent of cell surface expression of ICAM1. Collectively, these in vitro efficacy screening data support the notion that IC1-MMAE and IC1-MMAF are more potent ADC formulations than ICAM1-DM1 and ICAM1-DM4 for TNBC, warranting further investigation of these ADCs using in vivo TNBC models.

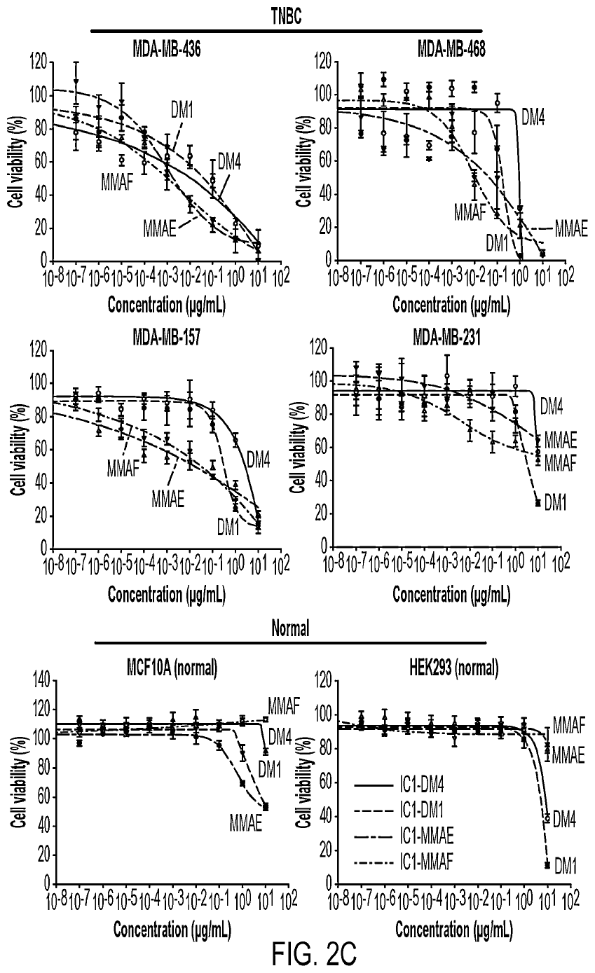

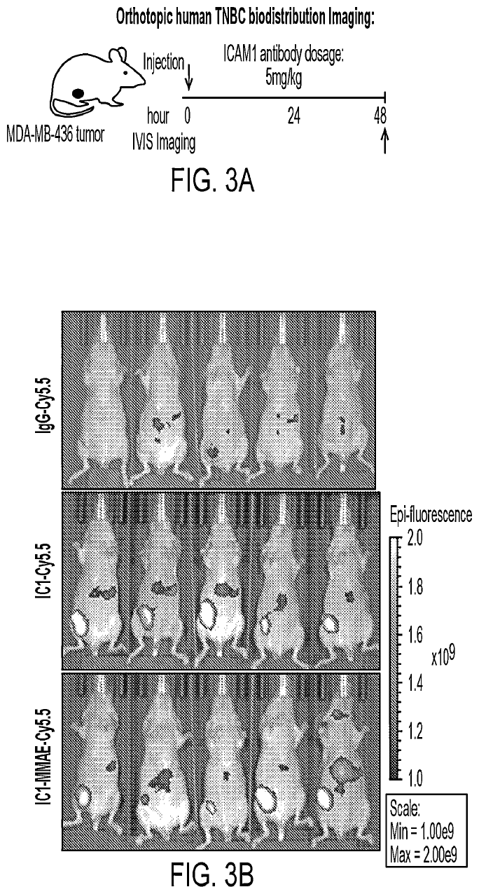

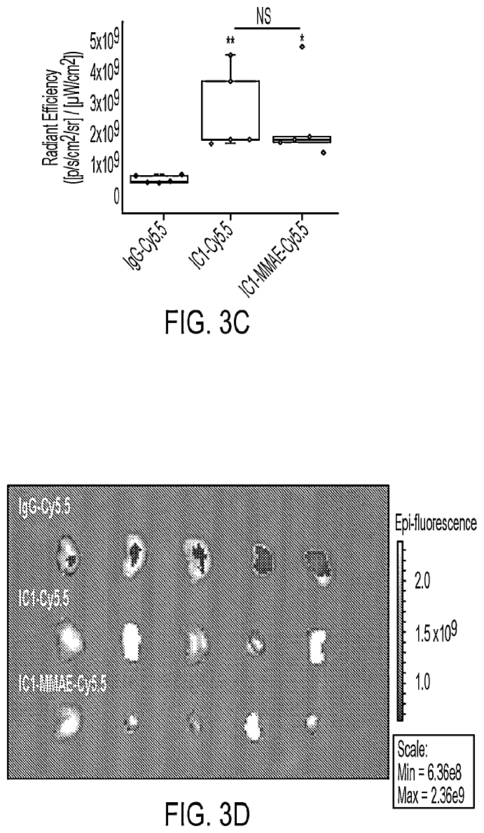

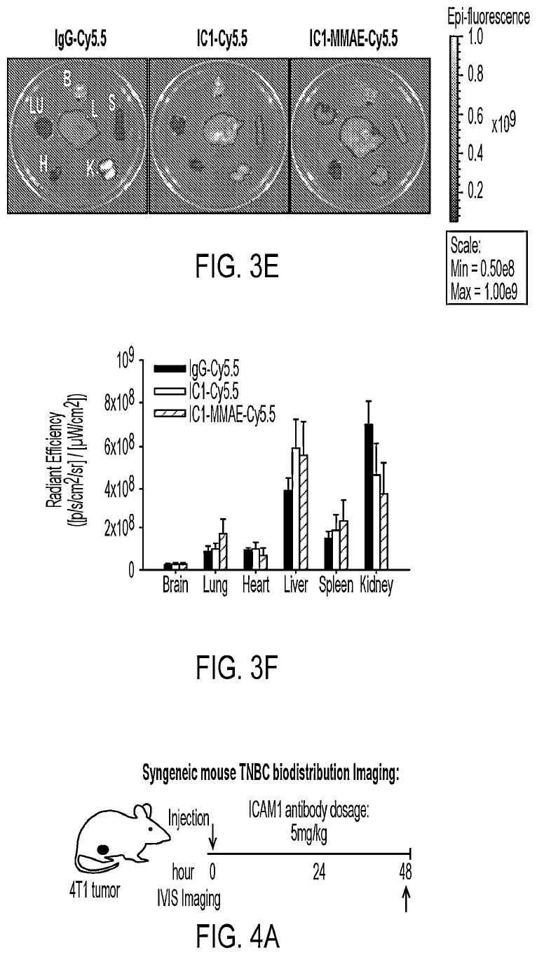

Next, the tumor specificity and biodistribution of ICAM1 ADCs was evaluated using both an immunocompromised nude mouse model with human TNBC tumors (MDA-MB-436) and an immunocompetent BALB/c mouse model with murine TNBC tumors (4T1), in order to investigate the effects of different immune system backgrounds. First, the nude mouse model was used to determine the tumor-specificity of ICAM1 antibody and ADC in orthotopic human TNBC tumors (FIG. 3A). Near-infrared fluorescent dye Cy5.5 labeled anti-human ICAM1 antibody (IC1-Cy5.5) and IC1-MMAE (IC1-MMAE-Cy5.5) were administered intravenously and compared to non-targeting IgG conjugated with Cy5.5 (IgG-Cy5.5) at an equivalent dosage of 5 mg/kg. At 24 hours post-injection, the nude mice were imaged by in vivo NIR fluorescent imaging. Mice treated with IC1-Cy5.5 and IC1-MMAE-Cy5.5 showed comparable intratumoral accumulation, approximately 5-fold higher than IgG-Cy5.5 (FIGS. 3B and 3D). These in vivo tumor accumulation data precisely matched the ex vivo NIR images of excised tumors (FIG. 3C). These results suggest that IC1-Cy5.5 and IC1-MMAE-Cy5.5 selectively recognize and bind human TNBC tumors in vivo, relative to non-targeting IgG-Cy5.5. The biodistribution of IC1-Cy5.5, IC1-MMAE-Cy5.5, and IgG-Cy5.5 was further examined in six major organs, namely in brain, lung, heart, liver, spleen, and kidney. Liver and kidney were the primary off-tumor accumulation sites of IC1-Cy5 and IC1-MMAE in tumor-bearing mice (FIGS. 3E and 3F). However, these are common off-tumor accumulation sites, and it was determined that any toxicities were well tolerated, based on serum biomarkers in subsequent animal studies.

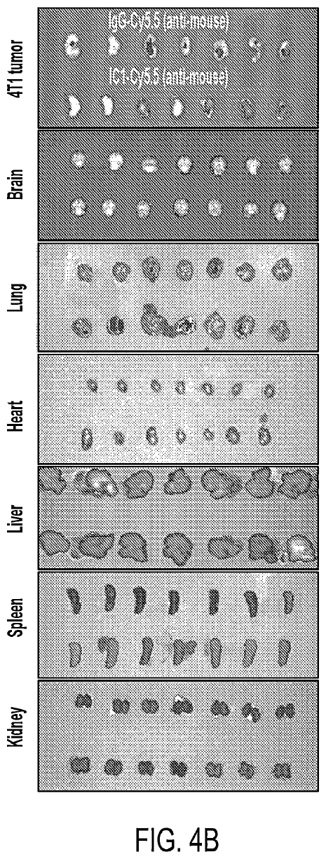

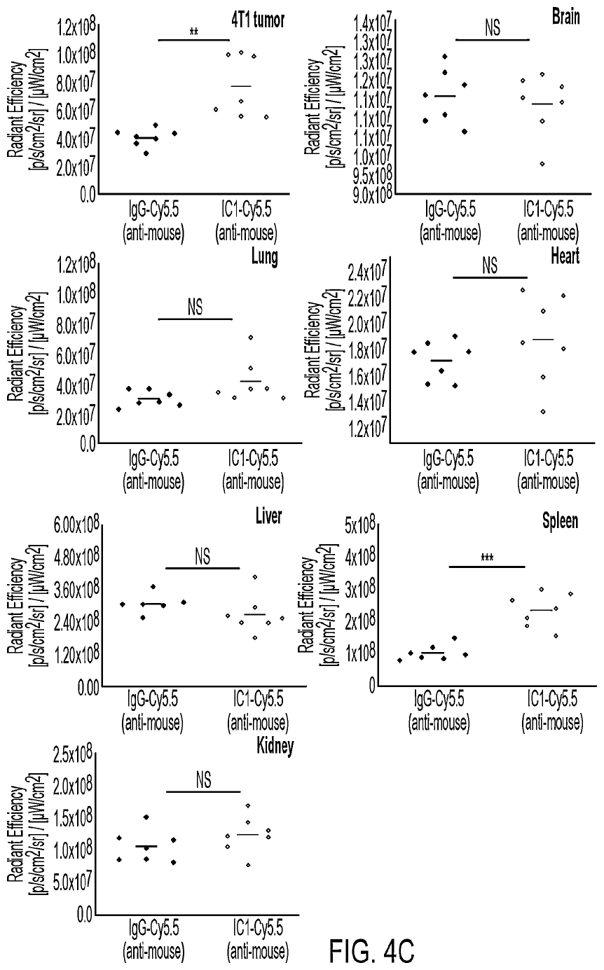

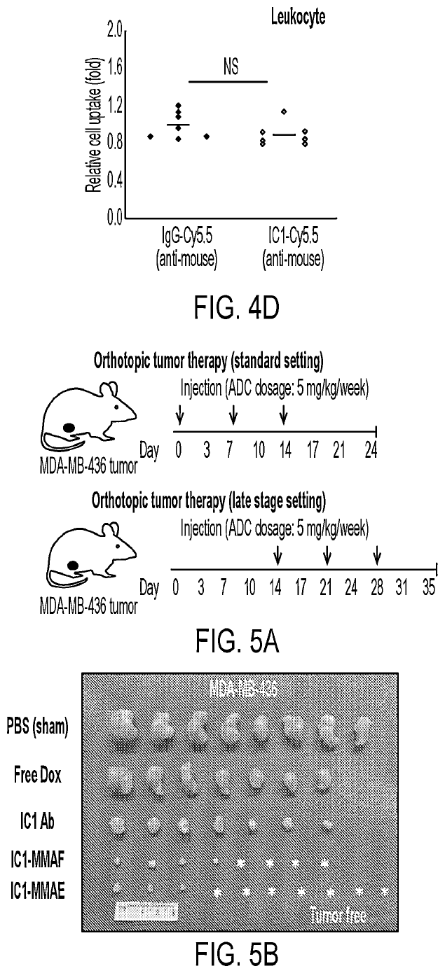

A potential concern for use of ICAM1 as a drug target for developing TNBC-targeted ADCs is its potential on-target/off-tumor toxicity in normal organs and tissues that normally express ICAM1. In the nude mouse model, anti-human ICAM1 antibodies cannot recognize mouse ICAM1 antigens expressed in normal mouse organs due to the lack of species cross-reactivity. To solve this issue, normal organ biodistribution was evaluated using anti-mouse ICAM1 antibodies in an immunocompetent BALB/c mouse model with murine 4T1 tumors (FIG. 4A). Notably, this biodistribution study was conducted in immunocompetent BALB/c mice which feature a complete immune system that can more faithfully recapitulate the interactions between the TNBC tumor microenvironment and ICAM1 antibodies, unlike the immunocompromised nude mouse model. Anti-mouse IC1-Cy5.5 maintains potent 4T1 tumor-specificity in BALB/c mice, in excellent correlation with anti-human IC1-Cy5.5 in nude mice (FIG. 4B). The biodistribution of anti-mouse IC1-Cy5.5 was examined in six normal organs relative to IgG-Cy5.5 (FIG. 4C). No preferential accumulation of anti-mouse IC1-Cy5.5 was observed in examined organs (i.e., brain, lung, heart, liver, and kidney), but was observed in the spleen. Additionally, the interaction between the immune system and ICAM1 antibodies was investigated by comparing the amount of IC1-Cy5.5 and IgG-Cy5.5 taken up by circulating leukocytes in tumor-bearing BALB/c mice. IC1-Cy5.5 showed the same level of circulating leukocyte uptake as IgG-Cy5.5, suggesting that ICAM1 antibodies do not target normal leukocytes in blood circulation (FIG. 4D).

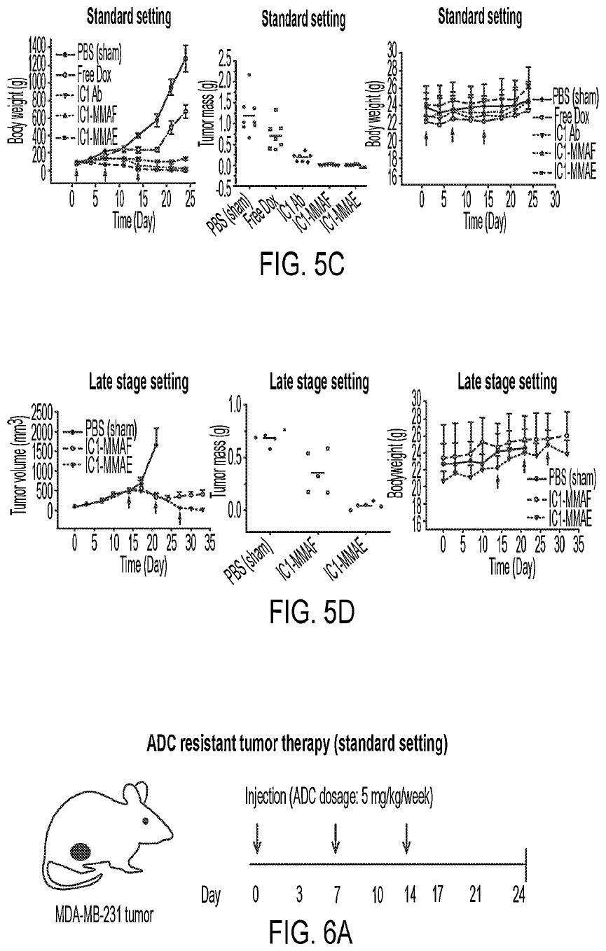

To evaluate the in vivo efficacy of IC1-MMAE and IC1-MMAF, a series of orthotopic TNBC models were assessed in standard, late-stage, and refractory tumor settings. The in vivo efficacy of IC1-MMAE or IC1-MMAF was first examined in the standard setting of an orthotopic TNBC model (MDA-MB-436) by initiating systemic IC1-MMAE or IC1-MMAF treatment at the dose of 5 mg/kg once the tumor reached a volume of 100 mm³ (FIG. 5A). Separate cohorts of tumor-bearing mice were treated with PBS (sham treatment), ICAM1 antibody alone (IC1 Ab), or doxorubicin (free Dox) as equivalent dosage controls. Both IC1-MMAE and IC1-MMAF elicited significant and durable tumor regression in every animal tested throughout the duration of the experiment (FIGS. 5B and 5C). The anti-tumor activities of IC1-MMAE and IC1-MMAF were determined by measuring tumor weights, which were found to be 99.5% and 99.0% less, respectively, that those of the PBS-treated group. More importantly, IC1-MMAE and IC1-MMAF successfully eradicated MDA-MB-436 tumors in 70% (7/10) and 50% (4/8) animals without tumor recurrence over a period of 28 days. In comparison, free Dox exhibited very limited efficacy against MDA-MB-436 tumors, while the IC1 Ab demonstrated a moderate anti-tumor efficacy, most likely by neutralizing ICAM1 signaling cascades in MDA-MB-436 tumors as previously reported by others²⁵⁻²⁷. IC1-Ab treatment was significantly less effective than IC1-MMAE and IC1-MMAF treatment, however.

To demonstrate the potential of IC1-MMAE and IC1-MMAF in combating even more aggressive TNBC tumors, the in vivo efficacy of IC1-MMAE and IC1-MMAF was evaluated in a late-stage setting of the same orthotopic TNBC model (MDA-MB-436). In the late-stage setting, MDA-MB-436 tumors were allowed to grow to a volume of 500 mm³ before systemic administration of IC1-MMAE or IC1-MMAF (FIG. 5A). IC1-MMAE significantly inhibited late-stage MDA-MB-436 tumor growth by 93.5% in all cases in comparison with the PBS-treated control group (FIG. 5D). While IC1-MMAF achieved 47.8% inhibition of tumor growth, this was significantly less effective than IC1-MMAE. Moreover, IC1-MMAE also eradicated late-stage MDA-MB-436 tumors in 20% (1/5) animals, while IC1-MMAF did not eradicate tumors. In both standard and late-stage settings, no weight loss was observed in animals treated with either IC1-MMAF or IC1-MMAE (FIGS. 5C and 5D), suggesting that these ICAM1 ADCs are well-tolerated in TNBC tumor-bearing nude mice at a dosage of 5 mg/kg.

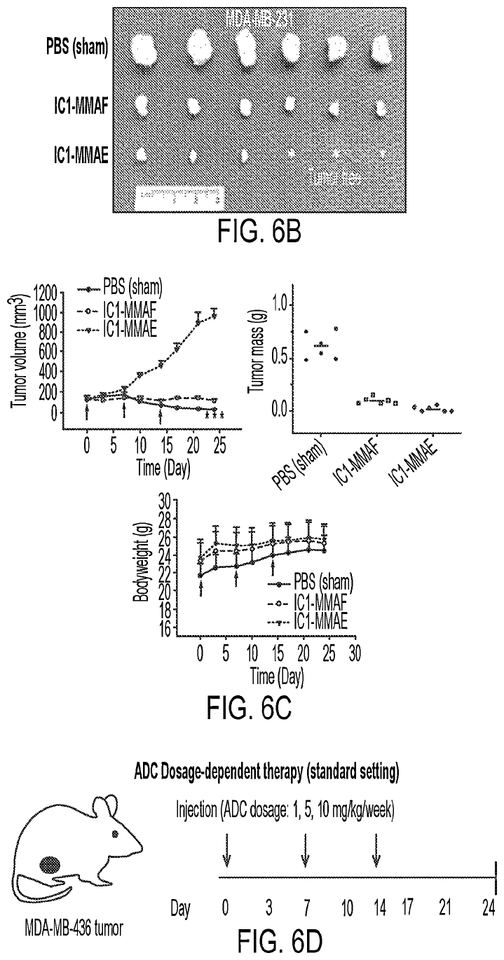

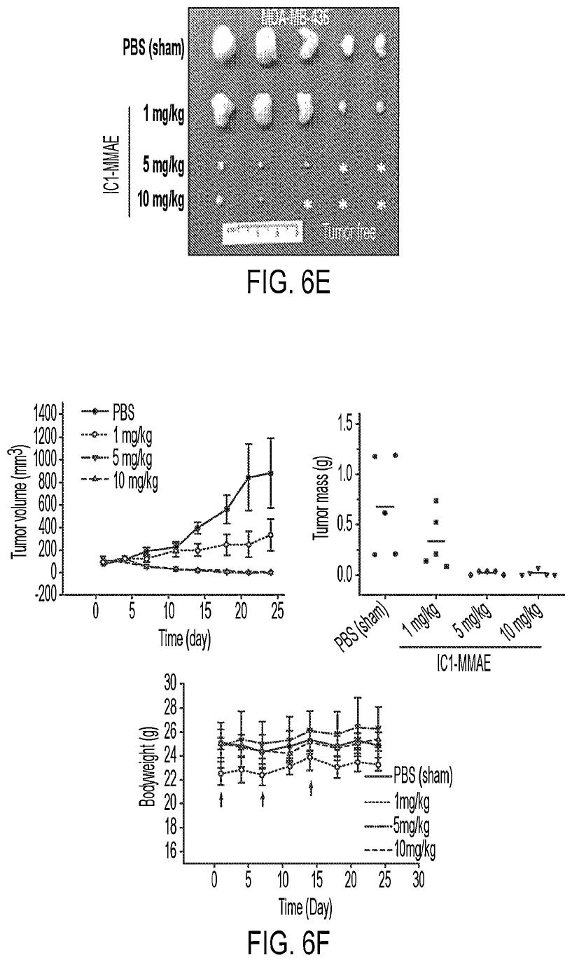

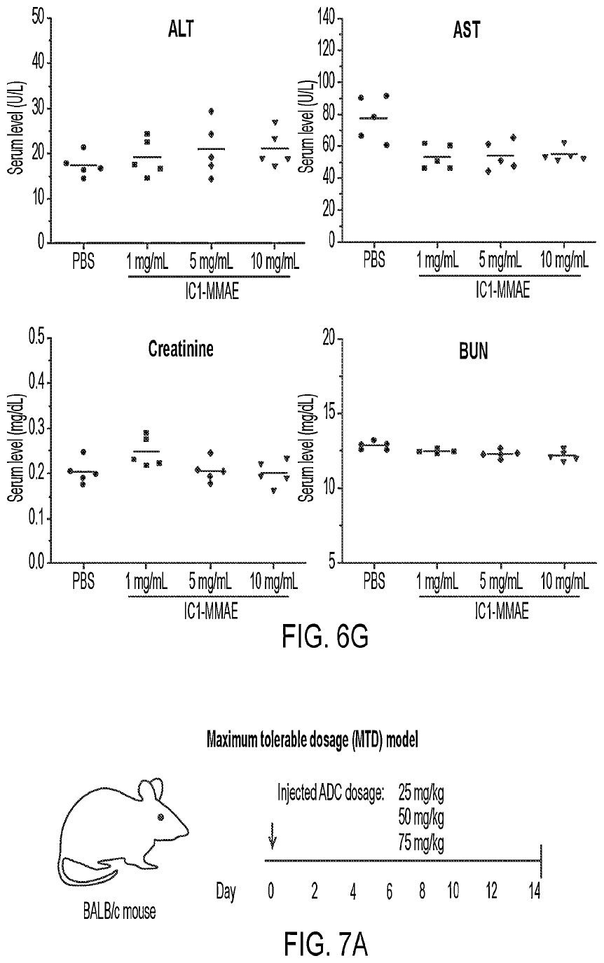

The in vivo efficacy of IC1-MMAE and IC1-MMAF was subsequently evaluated in refractory (ADC-resistant) TNBC tumors. In this study, the effect of ICAM1 ADCs on refractory TNBC tumors was examined by using ADC-resistant MDA-MB-231 cells previously established during the in vitro ADC efficacy test described previously. IC1-MMAE and IC1-MMAF were systemically administered at the same dosage of 5 mg/kg (FIG. 6A). Both IC1-MMAE and IC1-MMAF potently attenuated refractory MDA-MB-231 tumor growth by 96.7% and 84.3% respectively, in comparison with the PBS-treated control group (FIGS. 6B and 6C). IC1-MMAE also eradicated refractory tumors in 50% (3/6) animals, while IC1-MMAF did not eradicate tumors. Similarly, no body weight loss was observed during IC1-MMAE and IC1-MMAF treatment at the dose of 5 mg/kg in this refractory TNBC model. These data demonstrate that IC1-MMAE and IC1-MMAF are both effective in treating orthotopic TNBC tumors in the standard setting of targeted therapy, however IC1-MMAE is significantly more effective than IC1-MMAF for treating late-stage and refractory TNBC tumors. The higher efficacy of IC1-MMAE is likely due to its protease-cleavable MC-Vc-Pab linker, which mediates a faster mechanism of action than IC1-MMAF, which has a non-cleavable linker. Based on these in vivo efficacy results, IC1-MMAE was recognized as the optimal ICAM1 ADC formation for TNBC therapy.

Next, to determine the optimal dosage of IC1-MMAE for TNBC therapy, the in vivo efficacy of IC1-MMAE was evaluated in a dosage-dependent manner. In the standard setting of the orthotopic TNBC model (MDA-MB-436), three dosages of IC1-MMAE (1, 5, or 10 mg/kg) were tested alongside a PBS control (FIG. 6D). The anti-tumor activities of IC1-MMAE at 1, 5 and 10 mg/kg were determined to be 50.3%, 96.8% and 94.5%, respectively, as compared with the PBS-treated control group (FIGS. 6E and 6F). The tumor eradication rates for IC1-MMAE at 1, 5, 10 mg/kg were determined to be 0% (0/5), 40% (2/5) and 60% (3/5), respectively. These results suggest that IC1-MMAE administered at a dose of 5 mg/kg or higher is capable of eliciting significant and durable TNBC tumor regression and eradication in vivo. No body weight loss was observed as a consequence of IC1-MMAE at all tested dosages. Thus, 5 mg/kg was selected as the optimal dosage for IC1-MMAE treatment in pre-clinical animal models.

Any potential in vivo toxicity of IC1-MMAE at 1, 5 and 10 mg/kg dosages was also evaluated using standard blood chemistry analyses. After 24 days, blood samples from each group were collected via cardiac puncture and levels of aspartate aminotransferase (AST) and alanine aminotransferase (ALT), two established serum biomarkers of liver toxicity, were examined in sera from the blood samples. Even at the highest drug dosage (10 mg/kg), no elevation in either AST or ALT levels was observed relative to the PBS group (FIG. 6G). Similarly, renal toxicity of IC1-MMAE was assessed by measuring creatinine and blood urea nitrogen (BUN) levels. No renal toxicity was observed among the studied IC1-MMAE dosage groups (FIG. 6G).

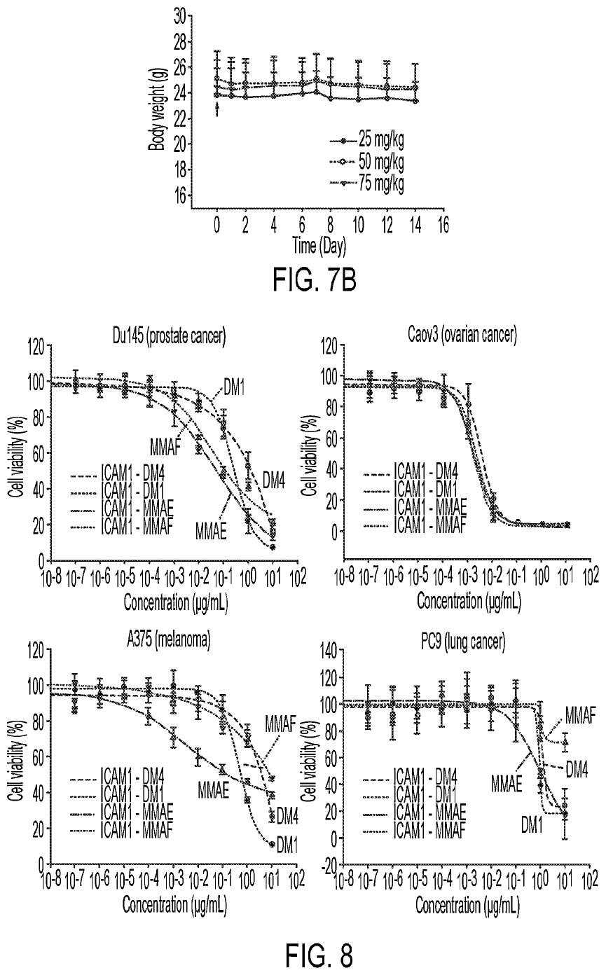

Finally, the maximum tolerated dose (MTD) of IC1-MMAE in healthy BALB/c mice was determined by intravenously administering IC1-MMAE at dosages of 25, 50 or 75 mg/kg, approximately 5 to 15-fold higher than IC1-MMAE dosage (5 mg/kg) used in the in vivo efficacy studies (FIG. 7A). No loss of body weight was observed in all IC-MMAE dosages tested, even at highest dosage of 75 mg/kg (FIG. 7B). Collectively, these in vivo toxicity studies strongly support the conclusion that IC1-MMAE is not only effective, but also well-tolerated as a TNBC treatment in pre-clinical in vivo models.

Beyond TNBC, the anti-tumor activity of four ICAM1 ADCs (ICAM1-DM4, ICAM1-DM1, ICAM1-MMAE, and ICAM1-MMAF) was examined against four additional human cancers that frequently express high levels of ICAM1 (FIG. 8). ICAM1 ADCs were observed to be effective in ablating each of these additional ICAM1-expressing human cancer cell lines, including prostate, ovarian, skin, and lung cancers. These results indicate that ICAM1 ADCs have the potential to be broadly used as precision therapeutics for the treatment of other human cancers that express significant levels of the ICAM1 antigen.

Together, these results provide the first experimental evidence of utilizing ICAM1 as an effective ADC target for TNBC. Due to the high level of ICAM1 expression on TNBC cells and low level of ICAM1 expression on healthy cells, ICAM1 ADCs can be used to specifically target cytotoxic drugs to TNBC cells while having minimal, if any, effect on non-cancer cells, as demonstrated. Alternate linkers and conjugated drugs for ICAM1 ADCs have also been evaluated as described herein. While multiple ADCs were found to be effective for treating TNBC, an ADC with a protease-cleavable linker and a microtubule inhibitor was found to be the optimal formulation for TNBC therapy among those tested, owing to its high and consistent efficacy for complete and durable TNBC tumor regression and eradication. The in vivo efficacy of this ADC (IC1-MMAE) is also higher than that of a clinically approved drug, sacituzumab govitecan, for treating refractory tumors in TNBC in vivo models³⁵, perhaps as a result of synergy between its higher TNBC-specificity and better optimized ADC formulation. The development of such an effective and well-tolerated therapeutic is promising for addressing the currently unmet need in TNBC treatment.

### Materials and Methods

### Materials

Phycoerythrin (PE)-conjugated anti-human ICAM1 antibody (Clone: HCD54), PE-conjugated mouse IgG isotype (PE-IgG), anti-mouse ICAM1 antibody (Clone #YN1/1.7.4) and RBC lysis buffer were purchased from BioLegend (San Diego, CA, USA). PE-conjugated anti-human TROP-2 antibody (Clone #77220), mouse and rat IgG isotype controls were purchased R&D systems (Minneapolis, MN, USA). Purified anti-human CD54 Antibody (Clone: R6.5), MC-VC-PAB-MMAE, MC-MMAF, Mal-EBE-MaL-DM1, Mal-EBE-Mal-DM4 were obtained from MabPlex (Yantai, China). Lab-Tek II Chamber Slide System was obtained from Thermo Fisher Scientific. Doxorubicin, bovine serum albumin (BSA), AST activity assay kit, ALT activity assay kit, creatinine activity assay kit, and urea activity assay kit were purchased from Sigma-Aldrich (St. Louis, MO). Dulbecco's PBS, Dulbecco's PBS, 4′,6-diamidino-2-phenylindole (DAPI), Gibco Dulbecco's modified Eagle's medium (DMEM), Gibco DMEM/F12(1:1) and Roswell Park Memorial Institute (RPMI)-1640 medium were purchased from Invitrogen (Carlsbad, CA). The Dojindo cell counting kit CCK-8 was purchased from Dojindo Molecular Technologies (Rockville, MD, USA).

### Cell Culture

Four human TNBC cell lines (MDA-MB-436, MDA-MB-468, MDA-MB-157, and MDA-MB-231), one murine TNBC cell line (4T1), non-neoplastic human mammary MCF10A cells and human embryonic kidney HEK293 cells were purchased from American Type Culture Collection (Manassas, VA, USA). MDA-MB-436, MDA-MB-468, MDA-MB-157, MDA-MB-231 and HEK293 cells were cultured in DMEM, 4T1 cells were cultured in RPMI-1640, and MCF10A cells were cultured in DMEM/F12 (1:1), with all recommended supplements. All cells were maintained at 37° C. in a humidified incubator with 5% CO₂.

### Genomic Analysis of ICAM1 Expression in TNBC Patients

The ICAM1 mRNA expression of human breast tumors and normal breast tissues were analyzed using the R2: Genomics Analysis and Visualization Platform database (hgserver1.amc.nl/). The following four datasheets were used in the genomic analysis: Tumor Breast Invasive Carcinoma-TCGA-1097; Tumor Breast (TNBC)-Brown-198; Tumor Breast compendium-Halfwerk-947; Tumor Breast (mutation status)-Meijers-Heijboer-155. The detailed information of microarray experiments and clinical samples are publically available in the R2: Genomics Analysis and Visualization Platform database.

### Flow Cytometry

1×10⁶ cells were collected and rinsed twice with PBS. Obtained cells were blocked by 1% BSA in PBS for 30 minutes in an ice bath. After BSA blockage, cells were incubated with PE-conjugated ICAM1 or TROP2 antibodies for 1 hour at room temperature, respectively. Non-targeting PE-conjugated IgG were used as a control. Cells were rinsed with 1% BSA in PBS three times, resuspended in PBS, and evaluated using a BD FACSCalibur Flow Cytometer (BD Biosciences, San Jose, CA, USA).

### Immunofluorescent Staining

2×10⁴ cells were seeded in a Lab-Tek II Chamber Slide System with 2 mL cell culture medium overnight at 37° C. After medium was removed, cells were rinsed twice with PBS and fixed with 4% formaldehyde in PBS at RT for 10 minutes, followed by washing with PBS. Samples were blocked with 1% BSA in PBS for 30 minutes in an ice bath. After BSA blocking, samples were co-stained with PE-conjugated ICAM1 or TROP2 antibody for 1 hour and rinsed with PBS. DAPI was used to stain cell nuclei. Immunofluorescent stained samples were dried overnight in the dark and then examined using a Leica TCS SP5 confocal fluorescent microscope (Leica Microsystems, Buffalo Grove, IL, USA).

### Imaging Flow Cytometry

1×10⁶ cells were seeded in each well of 6-well chamber slides and allowed to attach overnight. Then attached cells was incubated with PE-conjugated ICAM1 antibody for 1 hours in 2 mL cell culture media with 1% FBS at 37° C. Then the cell monolayer was collected and rinsed with cold PBS twice and resuspended. The cellular internalization rate of ICAM1 antibody in treated cells were evaluated using an Amnis imagestreamX Mark II imaging flow cytometry (Luminex, Austin, TX, USA).

### In Vitro Binding and Internalization of ICAM-1 Antibodies

The in vitro specific binding of ICAM1 antibody to the human TNBC cell lines (MDA-MB-231, MDA-MB-436, MDA-MB-157) was assessed using phycoerythrin (PE)-ICAM1 antibody. Non-cancerous MCF10A cells were used as the control. Cells were seeded in 8-well chamber slides at a density of 5×10³ cells/well. After recovering for 24 hours, the full media was replaced with that containing PE-ICAM1 antibody, with 1% FBS. Cells were incubated with the PE-ICAM1-containing media at 37° C. for an additional 4 hours. The cell monolayer was then rinsed with cold phosphate-buffered saline (PBS) and fixed with 4% paraformaldehyde in the PBS solution. Cell nuclei were counterstained with 4′,6′-diamidino-2-phenylindole hydrochloride (DAPI) using ProLong Gold Antifade Mountant. Fluorescence images were acquired and analyzed using a Zeiss LSM 880 confocal microscope (Oberkochen, Germany).

### Preparation and Characterization of ICAM1 ADCs

ICAM1-DM1, ICAM1-DM4, ICAM1-MMAE, and ICAM1-MMAF ADCs were prepared by conjugating ICAM1 antibody with Mal-EBE-Mal-DM1, Mal-EBE-Mal-DM4, MC-VC-PAB-MMAE, or MC-MMAF, respectively. The drug antibody ratios (DAR) of synthesized ADCs were characterized by hydrophobic interaction chromatography.

### In Vitro Efficacy and Cytotoxicity Assays

Human TNBC cell lines and control cells were seeded in a 96-well plate at a density of 5×10³ cells/well and allowed to adhere overnight. The culture medium was then replaced with medium containing IgG or ICAM1 conjugated to DM1, DM4, MMAE, or MMAF at different drug concentrations. After cells were cultured for another 48 hours, the cytotoxicity was determined by CCK-8 assay following the vendor-provided protocol. Cells were carefully rinsed with PBS after the drug-containing medium was removed, and this was followed by adding the CCK-8 containing medium solution. The cells were then incubated with the CCK-8 medium for 4 hours. The plate was read at the absorbance wavelength of 450 nm using a Molecular Devices SpectraMax microplate reader (San Jose, CA, USA). Cell viability was determined by comparing the absorbance of cells incubated with drugs to that of the control cells incubated without the presence of the drug.

### Orthotopic TNBC Mouse Models

Mouse studies presented in this study were performed according to the protocols approved by the Institutional Animal Care and Use Committee (IACUC) of Boston Children's Hospital. For tumor-specificity and in vivo biodistribution studies, orthotopic TNBC were implanted by injecting 2×10⁶ TNBC cells (MDA-MB-436 or 4T1) into the fourth right mammary fat pad of 6-8 weeks old female nude or BALB/c mice (Charles River, Wilmington, MA, USA). Tumors were allowed to develop for 2 to 3 weeks until they were at least 200-300 mm³ in volume, then tumor-bearing mice were randomized into various treatment groups (n≥5 per group) and received intravenously injection of IgG-Cy5.5, IC1-Cy5.5, or IC1-MMAE-Cy5.5 at a dosage of 5 mg/kg mouse weight. At 48 hours post-injection, in vivo NIR fluorescence imaging was performed on treated animals using an IVIS Lumina II system (Caliper, Hopkinton, MA, USA). Then mice were euthanized by CO₂ and 500 μL mouse blood was collected immediately via cardiac puncture. The NIR fluorescence intensities of various organs (brain, heart, liver, lung, kidney, and spleen) and excised tumors were measured using IVIS Lumina II. Mouse leukocytes were isolated from whole blood via centrifugation at 1200 rpm for 15 minutes and followed by removing red blood cells using RBC lysis buffer. The fluorescence intensity of IgG-Cy5.5 or IC1-Cy5.5 uptaken leukocytes were quantified using a BD FACSCalibur Flow Cytometer (BD Biosciences, San Jose, CA, USA).

For the in vivo efficacy studies, orthotopic TNBC tumors (MDA-MB-436 or MDA-MB-231) were established in 6-8 weeks old female nude mice as described above. Tumors were grown to reach 100 mm³ for standard setting treatment or 500 mm³ for late-stage treatment. Then tumor-bearing were randomly divided into various treatment groups (n≥5 per group) different groups and received treatment of PBS (sham), Free Dox, IC1 Ab, IC1-MMAE or IC1-MMAF at an equivalent dosage of 5 mg/kg per week for three weeks via tail vein injection. Tumor growth was monitored weekly using caliper. At the endpoint, animals were euthanized with CO₂ and orthotopic tumors were excised to measure their mass. In dosage-dependent experiments, three ascending IC1-MMAE dosages (1, 5, and 10 mg/kg) were evaluated in orthotopic TNBC tumors (MDA-MB-436) using the same protocol. Then mice were euthanized with CO₂ and 500 μL mouse blood was collected immediately via cardiac puncture. Collected blood was incubated for 20 minutes at room temperature to allow clotting and then mouse serum was isolated after centrifuging at 2,000×g for 10 minutes in a refrigerated centrifuge. Serum levels of ALT, AST, creatinine, and BUN were determined using their activity assay kits purchased from Sigma-Aldrich (St. Louis, MO, USA) with provided protocols. In MTD studies, the in vivo tolerability of IC1-MMAE were examined in 6-8 weeks old female BALB/c mice. Healthy mice were randomized into various treatment groups (n=10 per group) and received intravenously injection of IC1-MMAE at three ascending dosages (25, 50, and 75 mg/kg) for one injection. After injection, mouse bodyweight loss was used as the acute toxicity indicator and were closely monitored for up to 14 days.

### Statistical Analysis

All of the experimental data were obtained in triplicate and are presented as means±SD unless otherwise mentioned. One- and two-way analysis of variance (ANOVA) with Bonferroni post hoc tests were used to analyze statistical variance when making multiple comparisons. All statistical analysis was performed using OriginPro 8 software.

### Examples of the Structures of the Linker and Drug in the ADCs

The linker and drug structures used in the present disclosure are provided below.

### Name: Mal-EBE-Mal-DM1

### Chemical Structure:

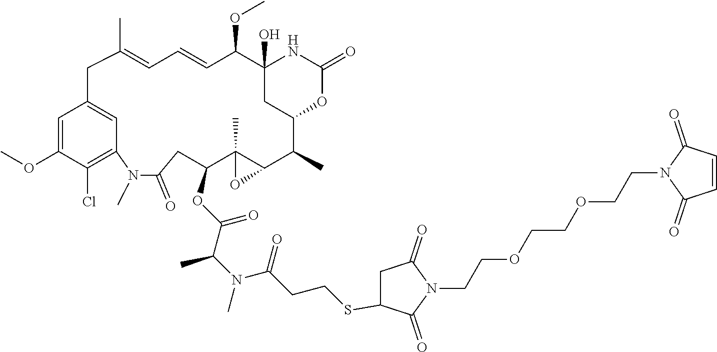 [SMILES: COc1cc2cc(c1Cl)N(C)C(=O)C[C@H](OC(=O)[C@H](C)N(C)C(=O)CCSC1CC(=O)N(CCOCCOCCN3C(=O)C=CC3=O)C1=O)[C@]1(C)O[C@H]1[C@H](C)[C@@H]1C[C@@](O)(NC(=O)O1)[C@H](OC)/C=C/C=C(\C)C2]

### Name: Mal-EBE-Mal-DM4

### Chemical Structure:

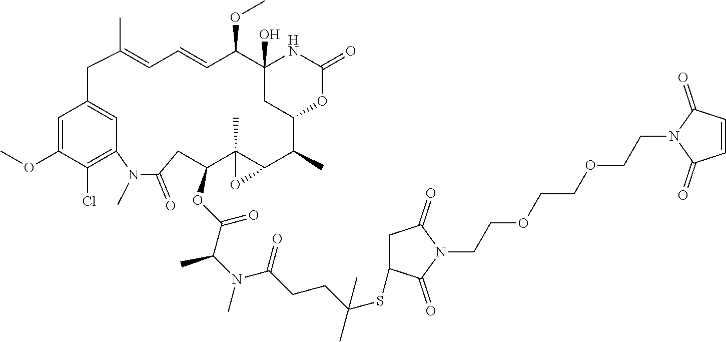 [SMILES: COc1cc2cc(c1Cl)N(C)C(=O)C[C@H](OC(=O)[C@H](C)N(C)C(=O)CCC(C)(C)SC1CC(=O)N(CCOCCOCCN3C(=O)C=CC3=O)C1=O)[C@]1(C)O[C@H]1[C@H](C)[C@@H]1C[C@@](O)(NC(=O)O1)[C@H](OC)/C=C/C=C(\C)C2]

### Name: MC-VC-PAB-MMAE

### Chemical Structure:

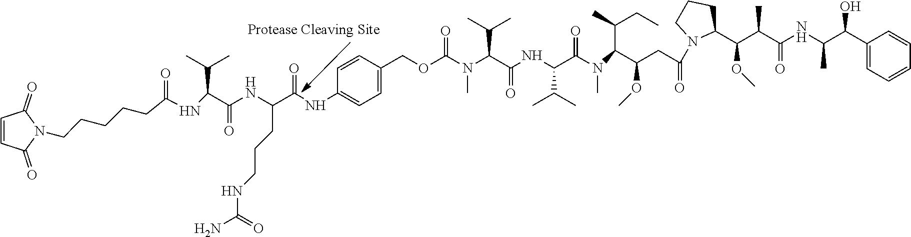 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)C(CCCNC(N)=O)NC(=O)[C@@H](NC(=O)CCCCCN2C(=O)C=CC2=O)C(C)C)cc1)C(C)C]

### Name: MC-MMAF

### Chemical Structure:

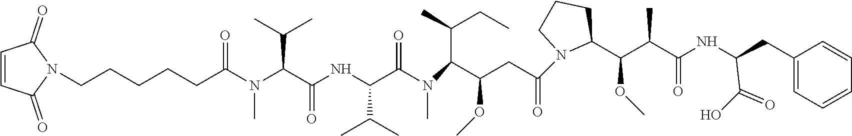 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C]

### References

1. R. L. Siegel, K. D. Miller, H. E. Fuchs, A. Jemal, Cancer Statistics, 2021, CA. Cancer J. Clin. 71, 7-33 (2021).
2. J. M. Lebert, R. Lester, E. Powell, M. Seal, J. McCarthy, Advances in the Systemic Treatment of Triple-Negative Breast Cancer, Curr. Oncol. 25, 142-150 (2018).
3. A. Lee, M. B. A. Djamgoz, Triple negative breast cancer: Emerging therapeutic modalities and novel combination therapies, Cancer Treat. Rev. 62, 110-122 (2018).
4. W. D. Foulkes, I. E. Smith, J. S. Reis-Filho, Triple-Negative Breast Cancer, N. Engl. J. Med. 363, 1938-1948 (2010).
5. R. Dent, M. Trudeau, K. I. Pritchard, W. M. Hanna, H. K. Kahn, C. A. Sawka, L. A. Lickley, E. Rawlinson, P. Sun, S. A. Narod, Triple-Negative Breast Cancer: Clinical Features and Patterns of Recurrence, Clin. Cancer Res. 13, 4429-4434 (2007).
6. M. Nedeljković, A. Damjanović, Mechanisms of Chemotherapy Resistance in Triple-Negative Breast Cancer—How We Can Rise to the Challenge, Cells 8, 957 (2019).
7. T. Ovcaricek, S. Frkovic, E. Matos, B. Mozina, S. Borstnar, Triple negative breast cancer—prognostic factors and survival, Radiol. Oncol. 45 (2011), doi:10.2478/v10019-010-0054-4.
8. D. Slamon, W. Eiermann, N. Robert, T. Pienkowski, M. Martin, M. Press, J. Mackey, J. Glaspy, A. Chan, M. Pawlicki, T. Pinter, V. Valero, M.-C. Liu, G. Sauter, G. von Minckwitz, F. Visco, V. Bee, M. Buyse, B. Bendahmane, I. Tabah-Fisch, M.-A. Lindsay, A. Riva, J. Crown, Adjuvant Trastuzumab in HER2-Positive Breast Cancer, N. Engl. J. Med. 365, 1273-1283 (2011).
9. K.-D. Yu, J. Wu, Z.-Z. Shen, Z.-M. Shao, Hazard of Breast Cancer-Specific Mortality among Women with Estrogen Receptor-Positive Breast Cancer after Five Years from Diagnosis: Implication for Extended Endocrine Therapy, J. Clin. Endocrinol. Metab. 97, E2201-E2209 (2012).
10. M. Robson, S.-A. Im, E. Senkus, B. Xu, S. M. Domchek, N. Masuda, S. Delaloge, W. Li, N. Tung, A. Armstrong, W. Wu, C. Goessl, S. Runswick, P. Conte, Olaparib for Metastatic Breast Cancer in Patients with a Germline BRCA Mutation, N. Engl. J. Med. 377, 523-533 (2017).
11. J. K. Litton, H. S. Rugo, J. Ettl, S. A. Hurvitz, A. Gonçalves, K.-H. Lee, L. Fehrenbacher, R. Yerushalmi, L. A. Mina, M. Martin, H. Roché, Y.-H. Im, R. G. W. Quek, D. Markova, I. C. Tudor, A. L. Hannah, W. Eiermann, J. L. Blum, Talazoparib in Patients with Advanced Breast Cancer and a Germline BRCA Mutation, N. Engl. J. Med. 379, 753-763 (2018).
12. P. Schmid, S. Adams, H. S. Rugo, A. Schneeweiss, C. H. Barrios, H. Iwata, V. Diéras, R. Hegg, S.-A. Im, G. Shaw Wright, V. Henschel, L. Molinero, S. Y. Chui, R. Funke, A. Husain, E. P. Winer, S. Loi, L. A. Emens, Atezolizumab and Nab-Paclitaxel in Advanced Triple-Negative Breast Cancer, N. Engl. J. Med. 379, 2108-2121 (2018).
13. C. H. Chau, P. S. Steeg, W. D. Figg, Antibody-drug conjugates for cancer, The Lancet 394, 793-804 (2019).
14. J. M. Lambert, A. Berkenblit, Antibody-Drug Conjugates for Cancer Treatment, Annu. Rev. Med. 69, 191-207 (2018).
15. M. J. Birrer, K. N. Moore, I. Betella, R. C. Bates, Antibody-Drug Conjugate-Based Therapeutics: State of the Science, JNCI J. Natl. Cancer Inst. 111, 538-549 (2019).
16. A. Bardia, I. A. Mayer, L. T. Vahdat, S. M. Tolaney, S. J. Isakoff, J. R. Diamond, J. O'Shaughnessy, R. L. Moroose, A. D. Santin, V. G. Abramson, N. C. Shah, H. S. Rugo, D. M. Goldenberg, A. M. Sweidan, R. Iannone, S. Washkowitz, R. M. Sharkey, W. A. Wegener, K. Kalinsky, Sacituzumab Govitecan-hziy in Refractory Metastatic Triple-Negative Breast Cancer, N. Engl. J. Med. 380, 741-751 (2019).
17. T. M. Bui, H. L. Wiesolek, R. Sumagin, ICAM-1: A master regulator of cellular responses in inflammation, injury resolution, and tumorigenesis, J. Leukoc. Biol. 108, 787-799 (2020).
18. P. Guo, J. Huang, L. Wang, D. Jia, J. Yang, D. A. Dillon, D. Zurakowski, H. Mao, M. A. Moses, D. T. Auguste, ICAM-1 as a molecular target for triple negative breast cancer, Proc. Natl. Acad. Sci. 111, 14710-14715 (2014).
19. P. Guo, J. Yang, D. Liu, L. Huang, G. Fell, J. Huang, M. A. Moses, D. T. Auguste, Dual complementary liposomes inhibit triple-negative breast tumor progression and metastasis, Sci. Adv. 5, eaav5010 (2019).
20. P. Guo, J. Yang, D. Jia, M. A. Moses, D. T. Auguste, ICAM-1-Targeted, Lcn2 siRNA-Encapsulating Liposomes are Potent Anti-angiogenic Agents for Triple Negative Breast Cancer, Theranostics 6, 1-13 (2016).
21. P. Guo, B. Wang, D. Liu, J. Yang, K. Subramanyam, C. R. McCarthy, J. Hebert, M. A. Moses, D. T. Auguste, Using Atomic Force Microscopy to Predict Tumor Specificity of ICAM1 Antibody-Directed Nanomedicines, Nano Lett. 18, 2254-2262 (2018).
22. P. Guo, J. Yang, J. Huang, D. T. Auguste, M. A. Moses, Therapeutic genome editing of triple-negative breast tumors using a noncationic and deformable nanolipogel, Proc. Natl. Acad. Sci. 116, 18295-18303 (2019).
23. B. Hoesel, J. A. Schmid, The complexity of NF-κB signaling in inflammation and cancer, Mol. Cancer 12, 86 (2013).
24. P. Melotti, E. Nicolis, A. Tamanini, R. Rolfini, A. Pavirani, G. Cabrini, Activation of NF-kB mediates ICAM-1 induction in respiratory cells exposed to an adenovirus-derived vector, Gene Ther. 8, 1436-1442 (2001).
25. C. L. Roland, A. H. Harken, M. G. San, C. C. Barnett, ICAM-1 expression determines malignant potential of cancer, Surgery 141, 705-707 (2007).
26. C. Rosette, R. B. Roth, P. Oeth, A. Braun, S. Kammerer, J. Ekblom, M. F. Denissenko, Role of ICAM1 in invasion of human breast cancer cells, Carcinogenesis 26, 943-950 (2005).
27. N. Veitonmäki, M. Hansson, F. Zhan, A. Sundberg, T. Löfstedt, A. Ljungars, Z.-C. Li, T. Martinsson-Niskanen, M. Zeng, Y. Yang, L. Danielsson, M. Kovacek, A. Lundqvist, L. Mårtensson, I. Teige, G. Tricot, B. Frendéus, A Human ICAM-1 Antibody Isolated by a Function-First Approach Has Potent Macrophage-Dependent Antimyeloma Activity In Vivo, Cancer Cell 23, 502-515 (2013).
28. Y. Bareche, D. Venet, M. Ignatiadis, P. Aftimos, M. Piccart, F. Rothe, C. Sotiriou, Unravelling triple-negative breast cancer molecular heterogeneity using an integrative multiomic analysis, Ann. Oncol. 29, 895-902 (2018).
29. on behalf of the German Consortium for Hereditary Breast and Ovarian Cancer (GC-HBOC), C. Engel, K. Rhiem, E. Hahnen, S. Loibl, K. E. Weber, S. Seiler, S. Zachariae, J. Hauke, B. Wappenschmidt, A. Waha, B. Blümcke, M. Kiechle, A. Meindl, D. Niederacher, C. R. Bartram, D. Speiser, B. Schlegelberger, N. Arnold, P. Wieacker, E. Leinert, A. Gehrig, S. Briest, K. Kast, O. Riess, G. Emons, B. H. F. Weber, J. Engel, R. K. Schmutzler, Prevalence of pathogenic BRCA1/2 germline mutations among 802 women with unilateral triple-negative breast cancer without family cancer history, BMC Cancer 18 (2018), doi:10.1186/s12885-018-4029-y.
30. B. N. Peshkin, M. L. Alabek, C. Isaacs, J. Eng-Wong, J. A. Zujewski, Eds. BRCA1/2 mutations and triple negative breast cancers, Breast Dis. 32, 25-33 (2011).
31. J. Li, X. Zhang, Z. Zhang, L. Zheng, S. Jindal, Y. Liu, Association of p53 expression with poor prognosis in patients with triple-negative breast invasive ductal carcinoma, Medicine (Baltimore) 98, e15449 (2019).
32. J. Huang, A. T. Agoston, P. Guo, M. A. Moses, A Rationally Designed ICAM1 Antibody Drug Conjugate for Pancreatic Cancer, Adv. Sci. 7, 2002852 (2020).
33. J. Huang, P. Guo, M. A. Moses, Rationally Designed Antibody Drug Conjugates Targeting the Breast Cancer-Associated Endothelium, ACS Biomater. Sci. Eng. 6, 2563-2569 (2020).
34. A. F. Kavanaugh, L. S. Davis, R. I. Jain, L. A. Nichols, S. H. Norris, P. E. Lipsky, A phase I/II open label study of the safety and efficacy of an anti-ICAM-1 (intercellular adhesion molecule-1; CD54) monoclonal antibody in early rheumatoid arthritis, J. Rheumatol. 23, 1338-1344 (1996).
35. D. M. Goldenberg, T. M. Cardillo, S. V. Govindan, E. A. Rossi, R. M. Sharkey, Trop-2 is a novel target for solid cancer therapy with sacituzumab govitecan (IMMU-132), an antibody-drug conjugate (ADC), Oncotarget 6, 22496-22512 (2015).
36. A. Suri, D. R. Mould, G. Song, J. Kinley, K. Venkatakrishnan, Population Pharmacokinetics of Brentuximab Vedotin in Adult and Pediatric Patients With Relapsed/Refractory Hematologic Malignancies: Model-Informed Hypothesis Generation for Pediatric Dosing Regimens, J. Clin. Pharmacol. 60, 1585-1597 (2020).

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of treating an intercellular adhesion molecule 1 (ICAM1)-expressing cancer, the method comprising administering to a subject in need thereof an effective amount of an antibody drug conjugate (ADC) comprising an ICAM1 antibody conjugated to a drug.

2. The method of claim 1, wherein the drug is selected from the group consisting of: N2′-Deacetyl-N2′-(3-mercapto-1-oxopropyl)mertansine (DM1), N2′-Deacetyl-N2′-(4-mercapto-4-methyl-1-oxopentyl)maytansine (DM4), monomethyl auristatin E (MMAE), monomethyl auristatin F (MMAF).

3. The method of claim 2, wherein the drug is MMAE.

4. The method of claim 2, wherein the drug is MMAF.

5. The method of any one of claims 1-4, wherein the ICAM1 antibody and the drug is conjugated via a linker.

6. The method of claim 5, wherein the linker is a cleavable linker.

7. The method of claim 6, wherein the cleavable linker is selected from the group consisting of: N-succinimidyl 4-(2-pyridyldithio)pentanoate (SPP), N-succinimidyl 3-(2-pyridyldithio)butanoate (SPDB), Sulfo-SPDB, valine-citrulline (Val-cit), acetyl butyrate, CL2A, and maleimidocaproyl (MC), and Mal-EBE-Mal.

8. The method of claim 5, wherein the linker is a non-cleavable linker.

9. The method of claim 8, wherein the non-cleavable linker is selected from the group consisting of: N-succinimidyl 4-(Nmaleimidomethyl)cyclohexane-1-carboxylate (SMCC) and maleimidomethyl cyclohexane-1-carboxylate (MCC), MC-VC-PAB.

10. The method of any one of claims 1-9, wherein the ICAM1 antibody is selected from the group consisting of an IgG, an Ig monomer, a Fab fragment, a F(ab′)2 fragment, a Fd fragment, a scFv, a scAb, a dAb, a Fv, an affibody, a diabody, a single domain heavy chain antibody, and a single domain light chain antibody.

11. The method of any one of claims 1-10, wherein the ICAM1 antibody is a chimeric or humanized antibody.

12. The method of any one of claims 1-11, wherein the ICAM1 antibody is R6.5 or HCD54.

13. The method of claim 12, wherein the ICAM1 antibody is a chimeric or humanized version of R6.5 or HCD54.

14. The method of any one of claims 1-13, wherein the ratio of the ICAM1 antibody and the drug in the ADC is 1:1 to 1:10.

15. The method of claim 14, wherein the ratio of the ICAM1 antibody and the drug in the ADC is 1:4.

16. The method of any one of claims 1-15, wherein the ADC is administered via injection.

17. The method of claim 16, wherein the injection is intravenous injection or intratumoral injection.

18. The method of any one of claims 1-17, wherein the ADC is administered via intravenous injection.

19. The method of any one of claims 1-18, wherein the ADC is administered at a dosage of 1 mg/kg to 75 mg/kg.

20. The method of claim 19, wherein the ADC is administered at a dosage of 5 mg/kg.

21. The method of any one of claims 1-20, wherein the ADC is administered from once every week to once every two months.

22. The method of any one of claims 1-21, wherein the subject is human.

23. The method of any one of claims 1-22, wherein the ICAM-1 expressing cancer is breast cancer, prostate cancer, ovarian cancer, melanoma, or lung cancer.

24. The method of claim 23, wherein the breast cancer is triple negative breast cancer (TNBC).

25. A method of treating triple negative breast cancer (TNBC), the method comprising administering to a subject in need thereof an effective amount of an antibody drug conjugate (ADC) comprising a chimeric intercellular adhesion molecule 1 (ICAM1) antibody conjugated to monomethyl auristatin E (MMAE) via a MC-VC-PAB linker.

26. A method of treating triple negative breast cancer (TNBC), the method comprising administering to a subject in need thereof an effective amount of an antibody drug conjugate (ADC) comprising a chimeric intercellular adhesion molecule 1 (ICAM1) antibody conjugated to monomethyl auristatin F (MMAF) via a MC linker.


SEQUENCE LISTING (2 sequences)

SEQ ID NO: 1 - protein, 537 aa, Mus musculus
MASTRAKPTLPLLLALVTVVIPGPGDAQVSIHPREAFLPQGGSVQVNCSSSCKEDLSLGLETQWLKDELESGPNWKLFELSEIGEDSSPLCFENCGTVQSSASATITVYSFPESVELRPLPAWQQVGKDLTLRCHVDGGAPRTQLSAVLLRGEEILSRQPVGGHPKDPKEITFTVLASRGDHGANFSCRTELDLRPQGLALFSNVSEARSLRTFDLPATIPKLDTPDLLEVGTQQKLFCSLEGLFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNNWTIIILVPVLLVIVGLVMAASYVYNRQRKIRIYKLQKAQEEAIKLKGQAPPP

SEQ ID NO: 2 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP